SEQUENCE LISTING (6 sequences)

SEQ ID NO: 1 - protein, 306 aa, Artificial Sequence
  2-amino-3-(4-(2-amino-3-mercaptopropanamido)phenyl)-propanoid acid aminoacyl tRNA synthetase.  (Derived from WT M. jannaschii tyrosyl tRNA synthetase.)
MDEFEMIKRNTSEIISEEELREVLKKDEKSAGIGFEPSGKIHLGHYLQIKKMIDLQNAGFDIIIDLADLIAYLNQKGELDEIREIGDYNKKVFEAMGLKAKYVYGSETYLDKDRTLNVYRLALKTTLKRARRSMELIAREDENPKVAEVIYPIMQVNGIHYEGVDVAVGGMEQRKIHMLARELLPKKVVCIHNPVLTGLDGEGKMSSSKGNFIAVDDSPEEIRAKIKKAYCPAGVVEGNPIMEIAKYFLGYPLTIKRPEKFGGDLTVNSYEELESLFKNKELHPMDLKNAVAEELIKILEPIRKRL

SEQ ID NO: 2 - DNA, 921 bp, Artificial Sequence
  2-amino-3-(4-(2-amino-3-mercaptopropanamido)phenyl)-propanoid acid aminoacyl tRNA synthetase.  (Derived from WT M. jannaschii tyrosyl tRNA synthetase.)
atggacgaatttgaaatgataaagagaaacacatctgaaattatcagcgaggaagagttaagagaggttttaaaaaaagatgaaaaatctgctgggataggttttgaaccaagtggtaaaatacatttagggcattatctccaaataaaaaagatgattgatttacaaaatgctggatttgatataattatagatttggctgatttaattgcctatttaaaccagaagggagagttggatgagattagagaaataggagattataacaaaaaagtttttgaagcaatggggttaaaggcaaaatatgtttatggaagtgaaacgtatcttgataaggatcgtacactgaatgtctatagattggctttaaaaactaccttaaaaagagcaagaaggagtatggaacttatagcaagagaggatgaaaatccaaaggttgctgaagttatctatccaataatgcaggttaatggtattcattatgagggcgttgatgttgcagttggagggatggagcagagaaaaatacacatgttagcaagggagcttttaccaaaaaaggttgtttgtattcacaaccctgtcttaacgggtttggatggagaaggaaagatgagttcttcaaaagggaattttatagctgttgatgactctccagaagagattagggctaagataaagaaagcatactgcccagctggagttgttgaaggaaatccaataatggagatagctaaatacttccttggatatcctttaaccataaaaaggccagaaaaatttggtggagatttgacagttaatagctatgaggagttagagagtttatttaaaaataaggaattgcatccaatggatttaaaaaatgctgtagctgaagaacttataaagattttagagccaattagaaagagattataa

SEQ ID NO: 3 - RNA, 77 nt, Methanococcus jannaschii
ugguccggcgggccggauuugaaccagcgccaugcggauuuagaguccgccguucugcccugcugaacuaccgccgg

SEQ ID NO: 4 - DNA, 25 bp, Artificial Sequence
  Oligonucleotide primer F53
cctgatatgaataaattgcagtttc

SEQ ID NO: 5 - DNA, 21 bp, Artificial Sequence
  Oligonucleotide primer F63
gttgtttacgctttgaggaat

SEQ ID NO: 6 - DNA, 17 bp, Artificial Sequence
  Oligonucleotide primer F67
gcggagcctatggaaaa

(19) United States
(12) Patent Application Publication
(10) Pub. No.: US 2011/0076718 A1
(43) Pub. Date: Mar. 31, 2011

(54) IN VIVO INCORPORATION OF AN UNNATURAL AMINO ACID COMPRISING A 1,2-AMINOTHIOL GROUP

(75) Inventors: Simon Ficht, San Diego, CA (US); Michael Jahnz, Berlin (DE); Jan Grunewald, San Diego, CA (US); Stefan Schiller, Pfaffenweiler (DE); Peter G. Schultz, La Jollla, CA (US)

(73) Assignee: The Scripps Research Institute

(21) Appl. No.: 12/735,966

(22) PCT Filed: Feb. 27, 2009
(86) PCT No.: PCT/US2009/001272

§ 371 Date: Nov. 15, 2010

Related U.S. Application Data
(60) Provisional application No. 61/067,524, filed on Feb. 27, 2008.

(51) Int. Cl. C12P 21/02; C12N 9/00; C07H 21/00

(52) U.S. Cl. 435/68.1; 435/183; 536/23.2

(57) ABSTRACT

The invention relates to orthogonal pairs of tRNAs and aminoacyl-tRNA synthetases that can incorporate unnatural amino acids that comprise a 1,2 aminothiol group into polypeptides. The invention provides translation systems in which polypeptides comprising unnatural amino acids that comprise a 1,2 aminothiol group can be produced. The invention also provides methods for producing polypeptides containing unnatural amino acids that comprise a 1,2 aminothiol group. Also provided by the invention are compositions comprising orthogonal aminoacyl-tRNA synthetases that preferentially aminoacylate a cognate orthogonal tRNA with unnatural amino acids that comprise a 1,2 aminothiol group. The invention provides methods for the synthesis of the unnatural amino acid 2-amino-3-(4-(2-amino-3-mercaptopropan-amido)phenyl)-propanoic acid.

### CROSS-REFERENCE TO RELATED APPLICATIONS

This application claims priority to and benefit of U.S. Provisional Patent Application Ser. No. 61/067,524, entitled, “IN VIVO INCORPORATION OF AN UNNATURAL AMINO ACID COMPRISING A 1,2-AMINOTHIOL GROUP,” by Simon Ficht, et al., filed Feb. 27, 2008, the contents of which are incorporated herein by reference in their entirety for all purposes.

### STATEMENT AS TO RIGHTS TO INVENTIONS MADE UNDER FEDERALLY SPONSORED RESEARCH AND DEVELOPMENT

The invention was made with United States Government support under Grant DE-FG02-03ER46051 from the Department of Energy. The United States Government has certain rights in the invention.

### FIELD OF THE INVENTION

The invention is in the field of translation biochemistry. The invention relates to compositions and methods for making and using orthogonal tRNAs, orthogonal aminoacyl-tRNA synthetases, and O-RS/O-tRNA pairs that incorporate unnatural amino acids that comprise a 1,2 aminothiol group into proteins. The invention also relates to methods of producing proteins comprising such unnatural amino acids in cells using such orthogonal pairs. In addition, the invention relates to proteins made by these methods.

### BACKGROUND OF THE INVENTION

Native chemical ligation (NCL) is a non-enzymatic, highly chemoselective reaction that proceeds efficiently in aqueous conditions at physiological pH. In the classical NCL reaction, a peptide comprising an N-terminal cysteine reacts with a moiety, e.g., another peptide, comprising an α-thioester group, e.g. a C-terminal thioester, in the presence of an exogenous thiol catalyst to yield a native peptide bond at the site of ligation (Dawson, et al. (1994) “Synthesis of Proteins by Native Chemical Ligation.” Science 266: 776-779). NCL can be used in protein semisynthesis (Schwarzer, et al. (2005) “Protein semisynthesis and expressed protein ligation: chasing a protein's tail.” Curr Op Chem Biol 9: 561-569) to generate cyclic peptides (Camarero, et al. (2001) “Peptide Chemical Ligation Inside Living Cells: In Vivo Generation of a Circular Protein Domain.” Bio Med Chem Lett 9: 2479-2484), to generate protein-liposome conjugates (Reulen, et al. (2007) “Protein-Liposome Conjugates Using Cysteine-Lipids And Native Chemical Ligation.” Bioconjugate Chem 18: 590-596), and to conjugate proteins to small molecule probes in vivo (Yeo, et al. (2003) “Cell-permeable small-molecule probes or site-specific labeling of proteins. Chem Commun 23: 2870-2871). NCL reactions have a variety of applications in biotechnology, biomedical research, and chemical biology, including peptide synthesis (Low, et al. (2001) “Total synthesis of cytochrome b562 by native chemical ligation using a removable auxiliary” Proc Natl Acad Sci USA 98: 6554-6559), chemical targeting of biomolecules in vivo (Yeo, et al. (2003) “Cell-permeable small-molecule probes or site-specific labeling of proteins. Chem Commun 23: 2870-2871), and immobilization of proteins on surfaces and resins (Girish, et al. (2005) “Site-specific immobilization of proteins in a microarray using intein-mediated protein splicing.” Bio Med Chem Lett 15: 2447-2451).

The requirement for an N-terminal cysteine residue is an intrinsic limitation of the NCL reaction. The native chemical ligation of peptides comprising N-terminal amino acids other than cysteine has been reported (WO98/28434; Canne, et al. (1996) “Extending the Applicability of Native Chemical Ligation.” J Am Chem Soc 118: 5891-5896), wherein the ligation is performed using a moiety comprising an α-thioester and a peptide or polypeptide segment having an N-terminal N-{thiol-substituted auxiliary} group represented by the formula HS—CH2—CH2—O—NH-[peptide]. Following ligation, the N-{thiol substituted auxiliary} group is removed by cleaving the HS—CH2—CH2—O-auxiliary group to generate a native peptide bond at the ligation site. However, this approach is suitable if a practitioner desires that the ligation product contain a glycine residue at the site of bond formation (Canne, et al. (1996) “Extending the Applicability of Native Chemical Ligation.” J Am Chem Soc 118: 5891-5896).

Alternately, removable N^{α}-(1-phenyl-2-mercaptoethyl)auxiliaries can be used to enable NCL reactions in polypeptides that do not contain cysteine residues (Botti, et al. (2001) “Native chemical ligation using removable N^{α}(1-phenyl-2-mercaptoethyl)auxiliaries.” Tetrahedron Lett 42: 1831-1833). In this approach, the (1-phenyl-2-mercaptoethyl)auxiliary on the α-amino group of a polypeptide of interest acts as a 1,2 aminothiol-containing functional group to effect thioester-mediated peptide bond-forming ligation with a second, α-thioester-containing moiety. Subsequent removal of the auxiliary from the newly formed peptide bond generates a ligation product that comprises a native peptide structure. Though this method enables ligation at a variety of amino acids and greatly increases the number of proteins accessible NCL, the formation of the peptide bond is nevertheless limited to the N-terminus of the peptide comprising the 1-phenyl-2-mercaptoethyl auxiliary moiety.

What are needed in the art are methods and compositions that permit NCL reactions at any desired amino acid position in a polypeptide. Recently, a general method was developed that makes it possible to genetically encode unnatural amino acids in both prokaryotic and eukaryotic organisms through the use of orthogonal tRNA/orthogonal aminoacyl tRNA synthetase pairs (reviewed in Wang and Schultz (2006) “Expanding the Genetic Code,” Ann Rev Biophys Biomol Struct 35: 225-249). This methodology has been successfully incorporate unnatural amino acids with unique chemical reactivities into proteins in bacteria and/or yeast (Chin, et al. (2002) “Addition of p-azido-L-phenylalanine to the genetic code of Escherichia coli.” J Am Chem Soc 124: 9026-9027; Deiters, et al. (2004) “Site-specific PEGylation of proteins containing unnatural amino acids.” Bio Med Chem Lett 14: 5743-5745; Wang, et al. (2003) “Addition of the keto functional group to the genetic code of Escherichia coli.” Proc Natl Acad Sci USA 100: 56-61; Zhang, et al. (2003) “A new strategy for the site-specific modification of proteins in vivo.” Biochemistry 42: 6735-46). There is a need in the art for unnatural amino acids that comprise a 1,2 aminothiol functional groups and for orthogonal translation components that can incorporate such unnatural amino acids at defined positions into proteins in living cells. The invention described herein fulfills these and other needs, as will be apparent upon review of the following disclosure.

### SUMMARY OF THE INVENTION

The invention provides systems, methods, compositions, and kits for incorporating unnatural amino acids that comprise a 1,2 aminothiol group, e.g., 2-amino-3-(4-(2-amino-3-mercaptopropanamido)phenyl)propanoic acid or other unnatural amino acids described herein, in response to a selector codon, e.g., an amber stop codon. These compositions include pairs of orthogonal tRNAs (O-tRNAs) and orthogonal aminoacyl tRNA synthetases (O-RSes) that do not interact with or interfere with the components of the translation system in which they are being used. These novel systems, methods, kits, and compositions permit the production of polypeptides comprising translationally incorporated unnatural amino acids that comprise a 1,2 aminothiol group, e.g., any of the unnatural amino acids described herein. Polypeptides that comprise such UAAs find particular use in native chemical ligation (NCL) reactions, where the 1,2 aminothiol moiety can readily and specifically react with a thioester moiety to form a native peptide bond. In addition, the 1,2 aminothiol moiety can readily and specifically react with an aldehyde moiety to form a thiazolidine. These reactions are highly chemoselective, proceed efficiently in aqueous conditions at physiological pH, and can be usefully applied to conjugate polypeptides to a wide range of target molecules, described elsewhere herein. Accordingly, compositions, methods, and systems for the site-specific incorporation of amino acids that comprise a 1,2 aminothiol group, e.g., the unnatural amino acids described herein, are a valuable tool for site-specific polypeptide modification, as demonstrated herein.

In one aspect, the invention provides translation systems. The translation systems comprise an unnatural amino acid that comprises a 1,2 aminothiol group, a first orthogonal aminoacyl-tRNA synthetase (O-RS), and a first orthogonal tRNA (O-tRNA), wherein the first O-RS preferentially aminoacylates the first O-tRNA with the unnatural amino acid that comprises a 1,2 aminothiol group. The unnatural amino acid comprising the 1,2 amino group can be, e.g., 2-amino-3-(4-(2-amino-3-mercaptopropanamido)phenyl)propanoic acid or any of the other unnatural amino acids discussed herein. In some aspects, the first O-RS preferentially aminoacylates the first O-tRNA with the unnatural amino acid that comprises a 1,2 aminothiol group with an efficiency that is at least 50% of the efficiency observed for a translation system comprising the first O-tRNA, the unnatural amino acid that comprises a 1,2 aminothiol group, and an aminoacyl-tRNA synthetase comprising the amino acid sequence of SEQ ID NO: 1.

The translation systems can use components derived from a variety of sources. In one embodiment, the O-RS used in the system can comprise an amino acid sequence of SEQ ID NO: 1 or a conservative variant thereof. The conservative variant can comprise a glycine at an amino acid position corresponding to amino acid 32 of SEQ ID NO: 1, an aspartic acid at an amino acid position corresponding to amino acid 65 of SEQ ID NO: 1, an isoleucine at an amino acid position corresponding to amino acid 70 of SEQ ID NO: 1, a glutamic acid at an amino acid position corresponding to amino acid 84 of SEQ ID NO: 1, a threonine at an amino acid position corresponding to amino acid 108 of SEQ ID NO: 1, a tyrosine at an amino acid position corresponding to amino acid 109 of SEQ ID NO: 1, an arginine at an amino acid position corresponding to amino acid 114 of SEQ ID NO: 1, a glycine at amino acid position corresponding to amino acid 158 of SEQ ID NO: 1, a glutamic acid at an amino acid position corresponding to amino acid 162 of SEQ ID NO: 1, and/or a glycine at an amino acid position corresponding to amino acid 250 of SEQ ID NO: 1.

In some embodiments, the O-tRNA can be an amber suppressor, an ochre suppressor tRNA, an opal suppressor tRNA, or a tRNA that recognizes a four base codon, a rare codon, or a non-coding codon. In some embodiments, the O-tRNA comprises or is encoded by the polynucleotide sequence of SEQ ID NO: 3.

In some aspects, the translation system optionally comprises a nucleic acid encoding a polypeptide of interest. This nucleic acid comprises at least one selector codon that is recognized by the O-tRNA. The polypeptide of interest encoded by the nucleic acid can comprise a Z-domain, an SH3 domain and/or any of the polypeptide domains discussed herein. The polypeptide of interest encoded by the nucleic acid can be homologous to c-Crk or any of the proteins discussed herein.

In some aspects, the translation system comprises a second orthogonal pair, e.g., a second O-RS and a second O-tRNA that utilize a second unnatural amino acid, so that the system is now able to incorporate at least two different unnatural amino acids at different selected sites in a polypeptide. In this embodiment, the second O-RS preferentially aminoacylates the second O-tRNA with a second unnatural amino acid that is different from the first unnatural amino acid, and the second O-tRNA recognizes a selector codon that is different from the selector codon recognized by the first O-tRNA.

In some embodiments, the translation system comprises a cell, e.g., a mammalian, an insect, a yeast, a bacterial, or an E. coli cell. The type of cell used is not particularly limited, as long as the O-RS and O-tRNA retain their orthogonality in the cell's environment.

Relatedly, the invention also provides methods, which use the translation systems described above, to produce polypeptides having one or more unnatural amino acids that comprise a 1,2 aminothiol group at selected positions. The polypeptides into which such unnatural amino acids can be incorporated are not particularly limited. The methods include providing a translation system comprising: i) an unnatural amino acid that comprises a 1,2, aminothiol group, e.g., 2-amino-3-(4-(2-amino-3-mercaptopropanamido)phenyl)propanoic acid or any one of the other unnatural amino acid described herein, ii) a first orthogonal aminoacyl-tRNA synthetase (O-RS), iii) a first orthogonal tRNA (O-tRNA) that preferentially aminoacylates the first O-tRNA with the unnatural amino acid that comprises an aminothiol group, and iv) a nucleic acid encoding a polypeptide, wherein the polynucleotide comprises at least one selector codon that is recognized by the first O-tRNA. The methods also include incorporating the unnatural amino acid that comprises a 1,2 aminothiol group at a selected position in the polypeptide during translation in response to the selector codon. In some embodiments, the unnatural amino acid is 2-amino-3-(4-(2-amino-3-mercaptopropanamido)phenyl)propanoic acid. Providing an O-RS can optionally include providing a nucleic acid that encodes the O-RS, where the nucleic acid comprises the polynucleotide sequence of SEQ ID NO: 2.

In some variations of these methods, the unnatural amino acid that comprises a 1,2 aminothiol group at the selected position in the polypeptide can be reacted with a moiety comprising an aldehyde functional group to form a thiazolidine. Optionally, the unnatural amino acid that comprises a 1,2 aminothiol group can be reacted with a moiety comprising a thioester functional group via native chemical ligation (NCL) to ligate the moiety to the polypeptide at the site of the unnatural amino acid with a peptide bond. The moiety comprising the aldehyde or thioester functional group can optionally be, e.g., a second amino acid in the polypeptide comprising the unnatural amino acid that comprises the 1,2 aminothiol group, a second translationally synthesized polypeptide, a second synthetic peptide, a second semi-synthetic peptide, an oligonucleotide, a DNA, an RNA, a nucleotide analog, an affinity tag (e.g., biotin, FLAG, hexahistine, etc.), a synthetic drug, a carbohydrate derivative, a fluorophore (e.g., Cascade Blue, Alexa568, Alexa647, etc.), a chromophore (e.g., phytochrome, phycobilin, bilirubin, etc.), a spin label (such as nitroxide), a toxin, a metal chelator (such as nitrilotriacetate), a photocrosslinker (such as p-azidoiodoacetanilide), an NMR probe, an X-ray probe, a pH probe, an IR probe, a dye, a sugar, a hapten, a cofactor, a fatty acid, a terpene (e.g., geraniol, limonene, farnesol, etc.), a polyethylene glycol (e.g., a branched PEG, a linear PEG, PEGs of different molecular weights, etc.), a resin, a solid support, or the like. It will be appreciated by those of skill in the art that the above list should not necessarily be taken as limiting

The invention also provides a variety of compositions, including nucleic acids and proteins. For example, the invention provides polynucleotides that encode an O-RS polypeptide that preferentially aminoacylates a cognate O-tRNA with an unnatural amino acid that comprises a 1,2 aminothiol group, e.g., those described herein. The O-RS polypeptide can comprise the amino acid sequence of SEQ ID NO: 1 or a conservative variant thereof. In some aspects, the conservative variant polypeptide can aminoacylate a cognate O-tRNA with 2-amino-3-(4-(2-amino-3-mercaptopropanamido)phenyl)propanoic acid with an efficiency that is at least 50% of the efficiency observed for a translation system comprising the cognate O-tRNA, the unnatural amino acid, and an aminoacyl-tRNA synthetase comprising the amino acid sequence of SEQ ID NO: 1.

The invention also provides polynucleotides that encode the O-RS polypeptides described above. For example, a polynucleotide can comprise the nucleotide sequence of SEQ ID NO: 2. Vectors and cells that comprise these polynucleotides are also provided by the invention.

Also provided by the invention are methods of producing an O-RS that preferentially aminoacylates an O-tRNA with a 2-amino-3-(4-(2-amino-3-mercaptopropanamido)phenyl)propanoic acid. The methods include mutating a wild-type aminoacyl-tRNA synthetase and selecting an O-RS mutant that preferentially aminoacylating an O-tRNA with 2-amino-3-(4-(2-amino-3-mercaptopropanamido)phenyl)propanoic acid.

In addition, the invention provides methods of synthesizing 2-amino-3-(4-(2-amino-3-mercaptopropanamido)phenyl)propanoic acid. The methods include dissolving N-(tert-butoxycarbonyl)-S-(triphenylmethyl)cysteine, 1-(3-dimethylaminopropyl)-3-ethylcarbodiimide hydrochloride, and 1-hydroxybenzotriazole hydrate in anhydrous dimethylformamide to produce solution 1, adding N,N-diisopropylethylamine to solution 1 to produce solution 2, and adding N-(tert-butoxycarbonyl)-4-aminophenylalanine to solution 2 to produce solution 3. The methods include drying solution 3 to produce residue 1, purifying residue 1 to produce solid 1, and dissolving solid 1 in a mixture comprising trifluoroacetic acid, triisopropylsilane, thioanisole and water to produce solution 4. The methods also include drying solution 4 to produce residue 2 and purifying residue 2, which comprises the 2-amino-3-(4-(2-amino-3-mercaptopropan-amido)phenyl)-propanoic acid.

Producing solution 2 can optionally comprise stirring solution 2 under argon for 5 minutes, producing solution 3 can comprise stirring solution 3 for 12 hours, and producing solution 4 can comprise stirring solution 4 for 20 minutes. Optionally, drying solution 3 to produce residue 1 and drying solution 4 to produce residue 2 can comprise vacuuming the solvent from each solution. Residue 1 can optionally be purified via column chromatography, and residue 2 can optionally be purified via HPLC to produce purified residue 2, which is then lyophilized to produce 2-amino-3-(4-(2-amino-3-mercaptopropan-amido)phenyl)-propanoic acid.

Kits are also a feature of the invention. For example, such kits can comprise components for producing a protein comprising one or more unnatural amino acids that comprise a 1,2 aminothiol group. Such components can include, e.g., a nucleic acid comprising a polynucleotide sequence encoding an O-tRNA, a nucleic acid comprising a polynucleotide encoding an O-RS, an O-RS, one or more unnatural amino acids comprising a 1,2 aminothiol group (such as those described herein), and/or reagents for the conjugation of the proteins comprising an unnatural amino acid with a 1,2 aminothiol group with a moiety comprising an aldehyde or thioester functional group (e.g., including, but not limited to those moieties describe above). The kits can optionally include a suitable strain of E. coli host cells for expression of the O-tRNA/O-RS and production of a protein comprising one or more unnatural amino acids that comprise a 1,2 aminothiol group. The kits can also include appropriate reagents and instructions for using polypeptides comprising unnatural amino acids with a 1,2 aminothiol group. Optionally, the kit can include reagents and instructions for the synthesis of 2-amino-3-(4-(2-amino-3-mercaptopropan-amido)phenyl)-propanoic acid. In addition, the kit can include a container to hold the kit components and/or instructional materials for practicing the methods herein with the compositions described above.

Those of skill in the art will appreciate that the methods, kits and compositions provided by the invention can be used alone or in combination. For example, a translation system of the invention can be used in the methods described herein to produce a polypeptide of interest comprising an unnatural amino acid with a 1,2 aminothiol group at a selected position. Alternately or additionally, these methods can be used to produce, e.g., proteins conjugated to one or more moieties (e.g., those described above) that comprise a thioester or aldehyde. One of skill will appreciate further combinations of the features of the invention noted herein.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a surface” includes a combination of two or more surfaces; reference to “bacteria” includes mixtures of bacteria, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

Bacteria: As used herein, the terms “bacteria” and “eubacteria” refer to prokaryotic organisms that are distinguishable from Archaea. Similarly, Archaea refers to prokaryotes that are distinguishable from eubacteria. Eubacteria and Archaea can be distinguished by a number morphological and biochemical criteria. For example, differences in ribosomal RNA sequences, RNA polymerase structure, the presence or absence of introns, antibiotic sensitivity, the presence or absence of cell wall peptidoglycans and other cell wall components, the branched versus unbranched structures of membrane lipids, and the presence/absence of histones and histone-like proteins are used to assign an organism to Eubacteria or Archaea.

Examples of Eubacteria include Escherichia coli, Thermus thermophilus, Bacillus subtilis and Bacillus stearothermophilus. Example of Archaea include Methanococcus jannaschii (Mj), Methanosarcina mazei (Mm), Methanobacterium thermoautotrophicum (Mt), Methanococcus maripaludis, Methanopyrus kandleri, Halobacterium such as Haloferax volcanii and Halobacterium species NRC-1, Archaeoglobus fulgidus (Af), Pyrococcus furiosus (Pf), Pyrococcus horikoshii (Ph), Pyrobaculum aerophilum, Pyrococcus abyssi, Sulfolobus solfataricus (Ss), Sulfolobtis tokodaii, Aeuropyrum pernix (Ap), Thermoplasma acidophilum and Thermoplasma volcanium.

Cognate: The term “cognate” refers to components that function together, or have some aspect of specificity for each other, e.g., an orthogonal tRNA and an orthogonal aminoacyl-tRNA synthetase.

Conservative variant: As used herein, the term “conservative variant,” in the context of a translation component, refers to a translation component, e.g., a conservative variant O-tRNA or a conservative variant O-RS, that functionally performs similar to a base component that the conservative variant is similar to, e.g., an O-tRNA or O-RS, having variations in the sequence as compared to a reference O-tRNA or O-RS. For example, an O-RS, or a conservative variant of that O-RS, will aminoacylate a cognate O-tRNA with an unnatural amino acid that comprises a 1,2 aminothiol group, e.g., 2-amino-3-(4-(2-amino-3-mercaptopropanamido)phenyl)propanoic acid. In this example, the O-RS and the conservative variant O-RS do not have the same amino acid sequences. The conservative variant can have, e.g., one variation, two variations, three variations, four variations, or five or more variations in sequence, as long as the conservative variant is still complementary to, e.g., functions with, the cognate corresponding O-tRNA or O-RS.

In some embodiments, a conservative variant O-RS comprises one or more conservative amino acid substitutions compared to the O-RS from which it was derived. In some embodiments, a conservative variant O-RS comprises one or more conservative amino acid substitutions compared to the O-RS from which it was derived, and furthermore, retains O-RS biological activity; for example, a conservative variant O-RS that retains at least 10% of the biological activity of the parent O-RS molecule from which it was derived, or alternatively, at least 20%, at least 30%, or at least 40%. In some preferred embodiments, the conservative variant O-RS retains at least 50% of the biological activity of the parent O-RS molecule from which it was derived. The conservative amino acid substitutions of a conservative variant O-RS can occur in any domain of the O-RS, including the amino acid binding pocket.

Derived from: As used herein, the term “derived from” refers to a component that is isolated from or made using a specified molecule or organism, or information from the specified molecule or organism. For example, a polypeptide that is derived from a second polypeptide can include an amino acid sequence that is identical or substantially similar to the amino acid sequence of the second polypeptide. In the case of polypeptides, the derived species can be obtained by, for example, naturally occurring mutagenesis, artificial directed mutagenesis or artificial random mutagenesis. The mutagenesis used to derive polypeptides can be intentionally directed or intentionally random, or a mixture of each. The mutagenesis of a polypeptide to create a different polypeptide derived from the first can be a random event, e.g., caused by polymerase infidelity, and the identification of the derived polypeptide can be made by appropriate screening methods, e.g., as discussed herein. Mutagenesis of a polypeptide typically entails manipulation of the polynucleotide that encodes the polypeptide.

Encode: As used herein, the term “encode” refers to any process whereby the information in a polymeric macromolecule or sequence string is used to direct the production of a second molecule or sequence string that is different from the first molecule or sequence string. As used herein, the term is used broadly, and can have a variety of applications. In some aspects, the term “encode” describes the process of semi-conservative DNA replication, where one strand of a double-stranded DNA molecule is used as a template to encode a newly synthesized complementary sister strand by a DNA-dependent DNA polymerase. In another aspect, the term “encode” refers to any process whereby the information in one molecule is used to direct the production of a second molecule that has a different chemical nature from the first molecule. For example, a DNA molecule can encode an RNA molecule, e.g., by the process of transcription incorporating a DNA-dependent RNA polymerase enzyme. Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

Eukaryote: As used herein, the term “eukaryote” refers to organisms belonging to the Kingdom Eucarya. Eukaryotes are generally distinguishable from prokaryotes by their typically multicellular organization (but not exclusively multicellular, for example, yeast), the presence of a membrane-bound nucleus and other membrane-bound organelles, linear genetic material (i.e., linear chromosomes), the absence of operons, the presence of introns, message capping and poly-A mRNA, and other biochemical characteristics, such as a distinguishing ribosomal structure. Eukaryotic organisms include, for example, animals, e.g., mammals, insects, reptiles, birds, etc., ciliates, plants, e.g., monocots, dicots, algae, etc., fungi, yeasts, flagellates, microsporidia, protists, etc.

Orthogonal: As used herein, the term “orthogonal” refers to a molecule, e.g., an orthogonal tRNA (O-tRNA) and/or an orthogonal aminoacyl-tRNA synthetase (O-RS), that functions with endogenous components of a cell with reduced efficiency as compared to a corresponding molecule that is endogenous to the cell or translation system, or that fails to function with endogenous components of the cell. In the context of tRNAs and aminoacyl-tRNA synthetases, orthogonal refers to an inability or reduced efficiency, e.g., less than 20% efficiency, less than 10% efficiency, less than 5% efficiency, or less than 1% efficiency, of an orthogonal tRNA to function with an endogenous tRNA synthetase compared to an endogenous tRNA to function with the endogenous tRNA synthetase, or of an orthogonal aminoacyl-tRNA synthetase to function with an endogenous tRNA compared to an endogenous tRNA synthetase to function with the endogenous tRNA. The orthogonal molecule lacks a functionally normal endogenous complementary molecule in the cell. For example, an orthogonal tRNA in a cell is aminoacylated by any endogenous RS of the cell with reduced or even zero efficiency, when compared to aminoacylation of an endogenous tRNA by the endogenous RS. In another example, an orthogonal RS aminoacylates any endogenous tRNA a cell of interest with reduced or even zero efficiency, as compared to aminoacylation of the endogenous tRNA by an endogenous RS. A second orthogonal molecule can be introduced into the cell that operably functions with the first orthogonal molecule. For example, an orthogonal tRNA/RS pair includes introduced complementary components that function together in the cell with an efficiency, e.g., 45% efficiency, 50% efficiency, 60% efficiency, 70% efficiency, 75% efficiency, 80% efficiency, 90% efficiency, 95% efficiency, or 99% or more efficiency, as compared to that of a control, e.g., a corresponding tRNA/RS endogenous pair, or an active orthogonal pair, e.g., an orthogonal tRNA/orthogonal RS pair.

Orthogonal aminoacyl-tRNA synthetase: As used herein, an orthogonal aminoacyl-tRNA synthetase (O-RS) is an enzyme that preferentially aminoacylates the O-tRNA with an amino acid in a translation system of interest. The amino acid that the O-RS loads onto the O-tRNA in the present invention is an unnatural amino acid comprising a 1,2 aminothiol group, e.g., 2-amino-3-(4-(2-amino-3-mercaptopropanamido)phenyl)propanoic acid or any of the unnatural amino acids described herein.

Orthogonal tRNA: As used herein, an orthogonal tRNA (O-tRNA) is a tRNA that is orthogonal to a translation system of interest. The O-tRNA can exist charged with, e.g., an unnatural amino acid that comprises a 1,2 aminothiol group, e.g., any of the unnatural amino acids shown in FIG. 1, or in an uncharged state. It is also to be understood that an O-tRNA is optionally charged (aminoacylated) by a cognate orthogonal aminoacyl-tRNA synthetase with an unnatural amino acid comprising a 1,2 aminothiol group. Indeed, it will be appreciated that the O-tRNA of the invention is advantageously used to insert an unnatural amino acid that comprises a 1,2 aminothiol group, e.g., any of the unnatural amino acids described herein, into a growing polypeptide, during translation, in response to a selector codon.

Preferentially aminoacylates: As used herein in reference to orthogonal translation systems, an O-RS “preferentially aminoacylates” a cognate O-tRNA when the O-RS charges the O-tRNA with, e.g., an unnatural amino acid that comprises a 1,2 aminothiol group, e.g., any of the unnatural amino acids depicted in FIG. 1, more efficiently than it charges any endogenous tRNA in an expression system. That is, when the O-tRNA and any given endogenous tRNA are present in a translation system in approximately equal molar ratios, the O-RS will charge the O-tRNA more frequently than it will charge the endogenous tRNA. Preferably, the relative ratio of O-tRNA charged by the O-RS to endogenous tRNA charged by the O-RS is high, preferably resulting in the O-RS charging the O-tRNA exclusively, or nearly exclusively, when the O-tRNA and endogenous tRNA are present in equal molar concentrations in the translation system. The relative ratio between O-tRNA and endogenous tRNA that is charged by the O-RS, when the O-tRNA and O-RS are present at equal molar concentrations, is greater than 1:1, preferably at least about 2:1, more preferably 5:1, still more preferably 10:1, yet more preferably 20:1, still more preferably 50:1, yet more preferably 75:1, still more preferably 95:1, 98:1, 99:1, 100:1, 500:1, 1,000:1, 5,000:1 or higher.

The O-RS “preferentially aminoacylates an O-tRNA with, e.g., an unnatural amino acid that comprises a 1,2 aminothiol group when (a) the O-RS preferentially aminoacylates the O-tRNA compared to an endogenous tRNA, and (b) where that aminoacylation is specific for e.g., the unnatural amino acid that comprises a 1,2 aminothiol group as compared to aminoacylation of the O-tRNA by the O-RS with any natural amino acid. That is, when e.g., the unnatural amino acid that comprises a 1,2 aminothiol group, e.g., any of the unnatural amino acids described herein, and natural amino acids are present in equal molar amounts in a translation system comprising the O-RS and O-tRNA, the O-RS will load the O-tRNA with e.g., the unnatural amino acid that comprises a 1,2 aminothiol group, e.g., 2-amino-3-(4-(2-amino-3-mercaptopropanamido)phenyl)propanoic acid, more frequently than with the natural amino acid. Preferably, the relative ratio of O-tRNA charged with e.g., the unnatural amino acid that comprises a 1,2 aminothiol group to O-tRNA charged with the natural amino acid is high. More preferably, O-RS charges the O-tRNA exclusively, or nearly exclusively, with e.g., the unnatural amino acid that comprises a 1,2 aminothiol group. The relative ratio between charging of the O-tRNA with, e.g., the unnatural amino acid that comprises a 1,2 aminothiol group and charging of the O-tRNA with a natural amino acid, when both the natural and the unnatural amino acid, e.g., any of the unnatural amino acids depicted in FIG. 1, are present in the translation system in equal molar concentrations, is greater than 1:1, preferably at least about 2:1, more preferably 5:1, still more preferably 10:1, yet more preferably 20:1, still more preferably 50:1, yet more preferably 75:1, still more preferably 95:1, 98:1, 99:1, 100:1, 500:1, 1,000:1, 5,000:1 or higher.

Prokaryote: As used herein, the term “prokaryote” refers to organisms belonging to the Kingdom Monera (also termed Procarya). Prokaryotic organisms are generally distinguishable from eukaryotes by their unicellular organization, asexual reproduction by budding or fission, the lack of a membrane-bound nucleus or other membrane-bound organelles, a circular chromosome, the presence of operons, the absence of introns, message capping and poly-A mRNA, and other biochemical characteristics, such as a distinguishing ribosomal structure. The Procarya include subkingdoms Eubacteria and Archaea (sometimes termed “Archaebacteria”). Cyanobacteria (the blue green algae) and mycoplasma are sometimes given separate classifications under the Kingdom Monera.

Selector codon: The term “selector codon” refers to codons recognized by the O-tRNA in the translation process and not recognized by an endogenous tRNA. The O-tRNA anticodon loop recognizes the selector codon on the mRNA and incorporates the amino acid with which it is charged, e.g., an unnatural amino acid that comprises a 1,2 aminothiol group, e.g., any of the unnatural amino acids shown in FIG. 1, at this site in the polypeptide. Selector codons can include, e.g., nonsense codons, such as, stop codons, e.g., amber, ochre, and opal codons; four or more base codons; rare codons; noncoding codons; and codons derived from natural or unnatural base pairs and/or the like.

Translation system: The term “translation system” refers to the components that incorporate an amino acid into a growing polypeptide chain (protein). Components of a translation system can include, e.g., ribosomes, tRNAs, synthetases, mRNA and the like. The O-tRNA and/or the O-RSs of the invention can be added to or be part of an in vitro or in vivo translation system, e.g., in a non-eukaryotic cell, e.g., a bacterium, such as E. coli, or in a eukaryotic cell, e.g., a yeast cell, a mammalian cell, a plant cell, an algae cell, a fungus cell, an insect cell, and/or the like.

Unnatural amino acid: As used herein, the term “unnatural amino acid” refers to any amino acid, modified amino acid, and/or amino acid analogue, that is not one of the 20 common naturally occurring amino acids. For example, unnatural amino acids that comprise 1,2 aminothiol groups, e.g., 2-amino-3-(4-(2-amino-3-mercaptopropanamido)phenyl)propanoic acid (see FIG. 1), find use with the invention. 2-amino-3-(4-(2-amino-3-mercaptopropanamido)phenyl)propanoic acid can also be called 4-(L-cysteinylamino)-L-phenylalanine. However, this unnatural amino acid will be referred to as 2-amino-3-(4-(2-amino-3-mercaptopropanamido)phenyl)propanoic acid throughout the present disclosure.

### BRIEF DESCRIPTION OF THE DRAWINGS

FIG. 1 depicts the chemical structure of 2-amino-3-(4-(2-amino-3-mercaptopropanamido)phenyl)propanoic acid and other unnatural amino acids that each comprise a 1,2 aminothiol group.

FIG. 2 depicts a reaction scheme for the synthesis of 2-(tert-butoxycarbonylamino)-3-(4-(2-(tert-butoxycarbonylamino)-3-(tritylthio)propanamido)phenyl)propanoic acid.

FIG. 3 depicts a reaction scheme for the synthesis of 2-amino-3-(4-(2-amino-3-mercaptopropanamido)phenyl)propanoic acid.

FIG. 4 depicts an SDS PAGE on which purified protein samples derived from cultures grown in the absence or presence 2-amino-3-(4-(2-amino-3-mercaptopropanamido)phenyl)propanoic acid were run.

FIG. 5 depicts the chemical structure of fluorescein-MES-thioester (C₂₃H₁₇O₉S₂⁻). Exact Mass: 501.03; Molecular Weight: 501.51; C. 55.08; H. 3.42; O. 28.71; S. 12.97.

FIG. 6 depicts an SDS PAGE on which samples of an NCL reaction mixture comprising the fluorescein-MES-thioester of FIG. 6 a Z-domain protein mutant comprising a 2-amino-3-(4-(2-amino-3-mercaptopropanamido)phenyl)propanoic acid residue at amino acid position 7 were run.

FIG. 7 depicts the SDS PAGE of FIG. 6 under UV light.

FIG. 8 depicts the reaction scheme for the site-specific PEGylation of a Z-domain protein mutant comprising a 2-amino-3-(4-(2-amino-3-mercaptopropanamido)phenyl)propanoic acid residue at amino acid position 7

FIG. 9 provides various nucleotide and amino acid sequences finding use with the invention.

FIG. 10 depicts results from experiments performed to ligate PEG-aldehydes to a Z-domain protein mutant comprising a 2-amino-3-(4-(2-amino-3-mercaptopropanamido)phenyl)propanoic acid residue at amino acid position 7 via native chemical ligation

### DETAILED DESCRIPTION

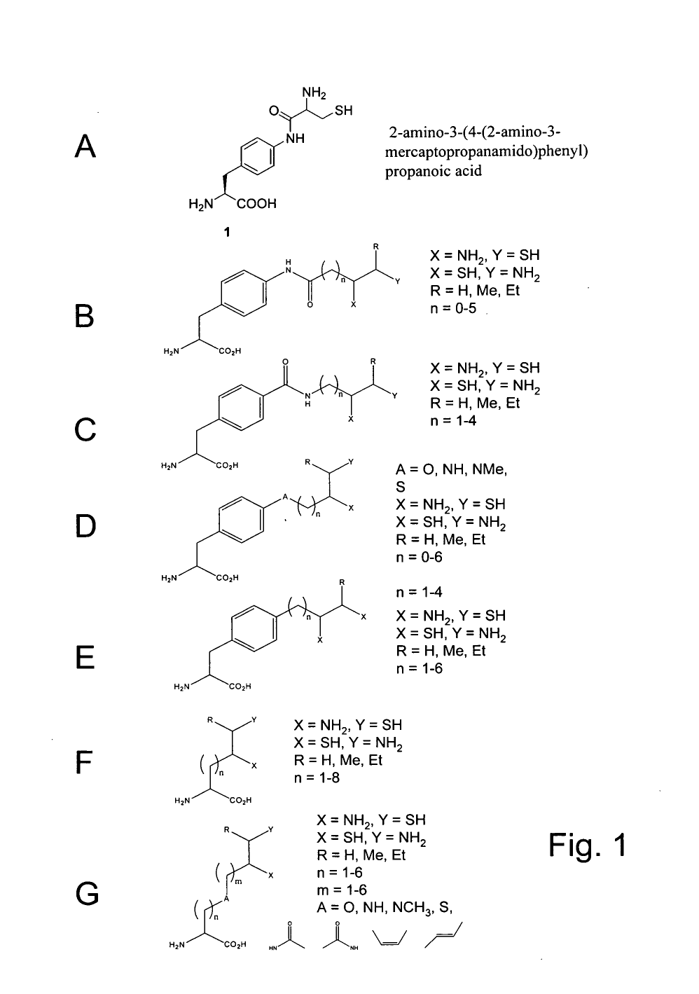

The invention described herein provides methods and compositions for the incorporation of unnatural amino acids that comprise a 1,2 aminothiol group (FIG. 1) into polypeptides using orthogonal translation systems. The incorporation of an unnatural amino acid that comprises a 1,2 aminothiol group, e.g., any of the unnatural amino acids described herein, into a polypeptide of interest can be programmed to occur at ay desired position by engineering the polynucleotide encoding the polypeptide of interest to contain a selector codon. The selector codon signals the incorporation of an unnatural amino acid that comprises a 1,2 aminothiol group, e.g., as shown in FIG. 1, into a specific position the primary structure of the growing polypeptide chain.

The novel compositions provided by the invention include a novel orthogonal aminoacyl-tRNA synthetases (O-RS) that have the ability to charge a suitable cognate suppressor orthogonal tRNA (O-tRNA), e.g., the O-tRNA of SEQ ID NO: 3, with an unnatural amino acid that comprises a 1,2 aminothiol group, e.g., 2-amino-3-(4-(2-amino-3-mercaptopropanamido)phenyl)propanoic acid. One example O-RS is a novel mutant of the Methanococcus jannaschii tyrosyl-tRNA synthetase and selectively charges the O-tRNA with, e.g., an unnatural amino acid that comprises a 1,2 aminothiol group, e.g., 2-amino-3-(4-(2-amino-3-mercaptopropanamido)phenyl)propanoic acid, in a translation system, e.g., in an E. coli cell. Most preferably, the O-RS and O-tRNA do not substantially cross-react with or interfere with the endogenous translational machinery of the translation system in which they are being used, e.g., the endogenous components of the translational machinery of, e.g., an E. coli cell or other host cell. The O-RS of the invention can include the O-RS of SEQ ID NO: 1. The invention also provides a polynucleotide that encodes this O-RS polypeptide, e.g., SEQ ID NO: 2.

The novel methods provided by the invention include steps for highly efficient and site-specific incorporation of, e.g., unnatural amino acids that comprise a 1,2 aminothiol group, e.g., any of the unnatural amino acids described herein, into polypeptides, e.g., in vivo, e.g., in an E. coli cell, in response to a selector codon, e.g., the amber nonsense codon TAG. These novel methods, as well as the novel compositions, can be used in, but are not limited to, a bacterial host system, e.g., E. coli.

The polypeptides into which the unnatural amino acids described herein, e.g., unnatural amino acids that comprise a 1,2 aminothiol group are incorporated can be efficiently modified under physiological conditions, e.g., in vitro and in vivo, in a highly selective fashion in native chemical ligation (NCL) reactions. In the classical NCL reaction, a peptide comprising an N-terminal cysteine reacts with a moiety, e.g., another peptide, comprising an a-thioester group, e.g. a C-terminal thioester, in the presence of an exogenous thiol catalyst to yield a native peptide bond at the site of ligation (Dawson, et al. (1994) “Synthesis of Proteins by Native Chemical Ligation.” Science 266: 776-779). The invention expands the utility of NCL by permitting the placement of the reactive 1,2 aminothiol at any amino acid position in an expressed polypeptide of interest, thus permitting the attachment of, e.g., a second amino acid in the polypeptide comprising the unnatural amino acid with the 1,2 aminothiol group, a second translationally synthesized polypeptide, a second synthetic peptide, a second semi-synthetic peptide, an oligonucleotide, a DNA, an RNA, a nucleotide analog, an affinity tag (e.g., biotin, FLAG, hexahistine, etc.), a synthetic drug, a carbohydrate derivative, a fluorophore (e.g., Cascade Blue, Alexa568, Alexa647, etc.), a chromophore (e.g., phytochrome, phycobilin, bilirubin, etc.), a spin label (such as nitroxide), a toxin, a metal chelator (such as nitrilotriacetate), a photocrosslinker (such as p-azidoiodoacetanilide), an NMR probe, an X-ray probe, a pH probe, an IR probe, a dye, a sugar, a hapten, a cofactor, a fatty acid, a terpene (e.g., geraniol, limonene, farnesol, etc.), a polyethylene glycol (e.g., a branched PEG, a linear PEG, PEGs of different molecular weights, etc.), a resin, a solid support, or the like, to an expressed polypeptide at any desired amino acid position. It will be appreciated by one of skill in the art that the list above should not be taken as limiting. Additionally or alternatively, an unnatural amino acid comprising a 1,2 aminothiol group that has been incorporated into a polypeptide of interest can be reacted with a moiety comprising an aldehyde, e.g., any one or more of the moieties described above, to conjugate the polypeptide to the moiety via a thiazolidine.

Orthogonal tRNA/Aminoacyl-tRNA Synthetase Technology

An understanding of the novel compositions and methods of the present invention is further developed through an understanding of the activities associated with orthogonal tRNA and orthogonal aminoacyl-tRNA synthetase pairs. In order to add additional unnatural amino acids that comprise a 1,2 aminothiol group to the genetic code, new orthogonal pairs comprising an aminoacyl-tRNA synthetase and a suitable tRNA are needed that can function efficiently in the host translational machinery, but that are “orthogonal” to the translation system at issue, meaning that O-RS/O-tRNA pair functions independently of the synthetases and tRNAs endogenous to the translation system. Desired characteristics of the orthogonal pair include a tRNA that decodes or recognizes only a specific codon, e.g., a selector codon, e.g., an amber stop codon, that is not decoded by any endogenous tRNA, and an aminoacyl-tRNA synthetase that preferentially aminoacylates, or “charges”, its cognate tRNA with only one specific unnatural amino acid. The O-tRNA is also not typically aminoacylated, or is poorly aminoacylated, i.e., charged, by endogenous synthetases. For example, in an E. coli host system, an orthogonal pair will include an aminoacyl-tRNA synthetase that does not cross-react with any of the endogenous tRNA, e.g., of which there are 40 endogenous in E. coli, and an orthogonal tRNA that is not aminoacylated by any of the endogenous synthetases, e.g., of which there are 21 in E. coli.

The general principles of orthogonal translation systems that are suitable for making proteins that comprise one or more unnatural amino acid are known in the art, as are the general methods for producing orthogonal translation systems. For example, see International Publication Numbers WO 2002/086075, entitled “METHODS AND COMPOSITION FOR THE PRODUCTION OF ORTHOGONAL tRNA-AMINOACYL-tRNA SYNTHETASE PAIRS;” WO 2002/085923, entitled “IN VIVO INCORPORATION OF UNNATURAL AMINO ACIDS;” WO 2004/094593, entitled “EXPANDING THE EUKARYOTIC GENETIC CODE;” WO 2005/019415, filed Jul. 7, 2004; WO 2005/007870, filed Jul. 7, 2004; WO 2005/007624, filed Jul. 7, 2004; WO 2006/110182, filed Oct. 27, 2005, entitled “ORTHOGONAL TRANSLATION COMPONENTS FOR THE VIVO INCORPORATION OF UNNATURAL AMINO ACIDS” and WO 2007/103490, filed Mar. 7, 2007, entitled “SYSTEMS FOR THE EXPRESSION OF ORTHOGONAL TRANSLATION COMPONENTS IN EUBACTERIAL HOST CELLS.” Each of these applications is incorporated herein by reference in its entirety. See also, e.g., Liu, et al. (2007) “Genetic incorporation of unnatural amino acids into proteins in mammalian cells” Nat Methods 4:239-244; and WO2006/110182 entitled “Orthogonal Translation Components for the In vivo Incorporation of Unnatural Amino Acids,” filed Oct. 27, 2005. For discussion of orthogonal translation systems that incorporate unnatural amino acids, and methods for their production and use, see also, Wang and Schultz, (2005) “Expanding the Genetic Code.” Angewandte Chemie Int Ed 44: 34-66; Xie and Schultz, (2005) “An Expanding Genetic Code.” Methods 36: 227-238; Xie and Schultz, (2005) “Adding Amino Acids to the Genetic Repertoire.” Curr Opinion in Chemical Biology 9: 548-554; and Wang et al., (2006) “Expanding the Genetic Code.” Annu Rev Biophys Biomol Struct 35: 225-249; Deiters, et al, (2005) “In vivo incorporation of an alkyne into proteins in Escherichia coli.” Bioorganic & Medicinal Chemistry Letters 15:1521-1524; Chin et al., (2002) “Addition of p-Azido-L-phenylalanine to the Genetic Code of Escherichia coli.” J Am Chem Soc 124: 9026-9027; and International Publication No. WO2006/034332, filed on Sep. 20, 2005, the contents of each of which are incorporated by reference in their entirety. Additional details are found in U.S. Pat. No. 7,045,337; U.S. Pat. No. 7,083,970; U.S. Pat. No. 7,238,510; U.S. Pat. No. 7,129,333; U.S. Pat. No. 7,262,040; U.S. Pat. No. 7,183,082; U.S. Pat. No. 7,199,222; and U.S. Pat. No. 7,217,809.

### Orthogonal Translation Systems

Orthogonal translation systems generally comprise cells, e.g., host cells such as E. coli, that include an orthogonal tRNA (O-tRNA), an orthogonal aminoacyl tRNA synthetase (O-RS), and an unnatural amino acid, e.g., an unnatural amino acid that comprises a 1,2 aminothiol group, e.g., those depicted in FIG. 1, wherein the O-RS aminoacylates the O-tRNA with the unnatural amino acid that comprises a 1,2 aminothiol group, e.g., 2-amino-3-(4-(2-amino-3-mercaptopropanamido)phenyl)propanoic acid. An orthogonal pair of the invention can include an O-tRNA, e.g., a suppressor tRNA, a frameshift tRNA, or the like, and a cognate O-RS. The orthogonal systems of the invention, which typically include O-tRNA/O-RS pairs, can comprise a cell or a cell-free environment. In addition to multi-component systems, the invention also provides novel individual components, for example, a novel orthogonal aminoacyl-tRNA synthetase polypeptide, e.g., SEQ ID NO: 1, and the polynucleotide that encodes that polypeptide, e.g., SEQ ID NO: 2.

In general, when an orthogonal pair recognizes a selector codon and loads an amino acid in response to the selector codon, the orthogonal pair is said to “suppress” the selector codon. That is, a selector codon that is not recognized by the translation system's, e.g., the E. coli cell's, endogenous machinery is not ordinarily charged, which results in blocking production of a polypeptide that would otherwise be translated from the nucleic acid. In an orthogonal pair system, the O-RS aminoacylates the O-tRNA with a specific unnatural amino acid, e.g., an unnatural amino acid that comprises a 1,2 aminothiol group, e.g., any of the unnatural amino acids described herein. The charged O-tRNA recognizes the selector codon and suppresses the translational block caused by the selector codon.

In some aspects, an O-tRNA of the invention recognizes a selector codon and includes at least about, e.g., a 45%, a 50%, a 60%, a 75%, a 80%, or a 90% or more suppression efficiency in the presence of a cognate synthetase in response to a selector codon as compared to the suppression efficiency of an O-tRNA comprising or encoded by a polynucleotide sequence as set forth in the sequence listing herein.

In some embodiments, the suppression efficiency of the O-RS and the O-tRNA together is about, e.g., 5 fold, 10 fold, 15 fold, 20 fold, or 25 fold or more greater than the suppression efficiency of the O-tRNA lacking the O-RS. In some aspect, the suppression efficiency of the O-RS and the O-tRNA together is at least about, e.g., 35%, 40%, 45%, 50%, 60%, 75%, 80%, or 90% or more of the suppression efficiency of an orthogonal synthetase pair as set forth in the sequence listings herein.

The translation system, e.g., an E. coli cell, uses the O-tRNA/O-RS pair to incorporate the unnatural amino acid that comprises a 1,2 aminothiol group into a growing polypeptide chain, e.g., via a nucleic acid that comprises a polynucleotide that encodes a polypeptide of interest, where the polynucleotide comprises a selector codon that is recognized by the O-tRNA. In certain preferred aspects, the cell can include one or more additional O-tRNA/O-RS pairs, where the additional O-tRNA is loaded by the additional O-RS with a different unnatural amino acid. For example, one of the O-tRNAs can recognize a four base codon and the other O-tRNA can recognize a stop codon. Alternately, multiple different stop codons, multiple different four base codons, multiple different rare codons and/or multiple different non-coding codons can be used in the same coding nucleic acid. For further details regarding available O-RS/O-tRNA cognate pairs and their use, see, e.g., the references noted above.

As noted, in some embodiments, there exist multiple O-tRNA/O-RS pairs in translation system, which allow incorporation of more than one unnatural amino acid into a polypeptide. For example, the translation system can further include an additional different O-tRNA/O-RS pair and a second unnatural amino acid, where this additional O-tRNA recognizes a second selector codon and this additional O-RS preferentially aminoacylates the O-tRNA with the second unnatural amino acid. For example, a cell that includes an O-tRNA/O-RS pair, where the O-tRNA recognizes, e.g., an amber selector codon, can further comprise a second orthogonal pair, where the second O-tRNA recognizes a different selector codon, e.g., an opal codon, an ochre codon, a four-base codon, a rare codon, a non-coding codon, or the like. Desirably, the different orthogonal pairs are derived from different sources, which can facilitate recognition of different selector codons.

In certain embodiments, translation systems can comprise a cell, such as an E. coli cell, that includes an orthogonal tRNA (O-tRNA), an orthogonal aminoacyl-tRNA synthetase (O-RS), an unnatural amino acid that comprises a 1,2 aminothiol group, e.g., any of the unnatural amino acids shown in FIG. 1, and a nucleic acid that comprises a polynucleotide that encodes a polypeptide of interest, where the polynucleotide comprises the selector codon that is recognized by the O-tRNA. Although orthogonal translation systems, e.g., translation systems comprising an O-RS, an O-tRNA and an unnatural amino acid that comprises a 1,2 aminothiol group, can utilize cultured cells to produce proteins having unnatural amino acids, it is not intended that an orthogonal translation system of the invention require an intact, viable cell. For example, a orthogonal translation system can utilize a cell-free system in the presence of a cell extract. Indeed, the use of cell free, in vitro transcription/translation systems for protein production is a well established technique. Adaptation of these in vitro systems to produce proteins having unnatural amino acids using orthogonal translation system components described herein is well within the scope of the invention.

The O-tRNA and/or the O-RS can be naturally occurring or can be, e.g., derived by mutation of a naturally occurring tRNA and/or RS, e.g., by generating libraries of tRNAs and/or libraries of RSs, from any of a variety of organisms and/or by using any of a variety of available mutation strategies. For example, one strategy for producing an orthogonal tRNA/aminoacyl-tRNA synthetase pair involves importing a tRNA/synthetase pair that is heterologous to the system in which the pair will function from a source, or multiple sources, other than the translation system in which the tRNA/synthetase pair will be used. The properties of the heterologous synthetase candidate include, e.g., that it does not charge any host cell tRNA, and the properties of the heterologous tRNA candidate include, e.g., that it is not aminoacylated by any host cell synthetase. In addition, the heterologous tRNA is orthogonal to all host cell synthetases. A second strategy for generating an orthogonal pair involves generating mutant libraries from which to screen and/or select an O-tRNA or O-RS. These strategies can also be combined.

Orthogonal tRNA (O-tRNA)

An orthogonal tRNA (O-tRNA) of the invention desirably mediates incorporation of an unnatural amino acid into a protein that is encoded by a polynucleotide that comprises a selector codon that is recognized by the O-tRNA, e.g., in vivo or in vitro. In certain embodiments, an O-tRNA of the invention includes at least about, e.g., a 45%, a 50%, a 60%, a 75%, a 80%, or a 90% or more suppression efficiency in the presence of a cognate synthetase in response to a selector codon as compared to an O-tRNA comprising or encoded by a polynucleotide sequence as set forth in the O-tRNA sequences in the sequence listing herein.

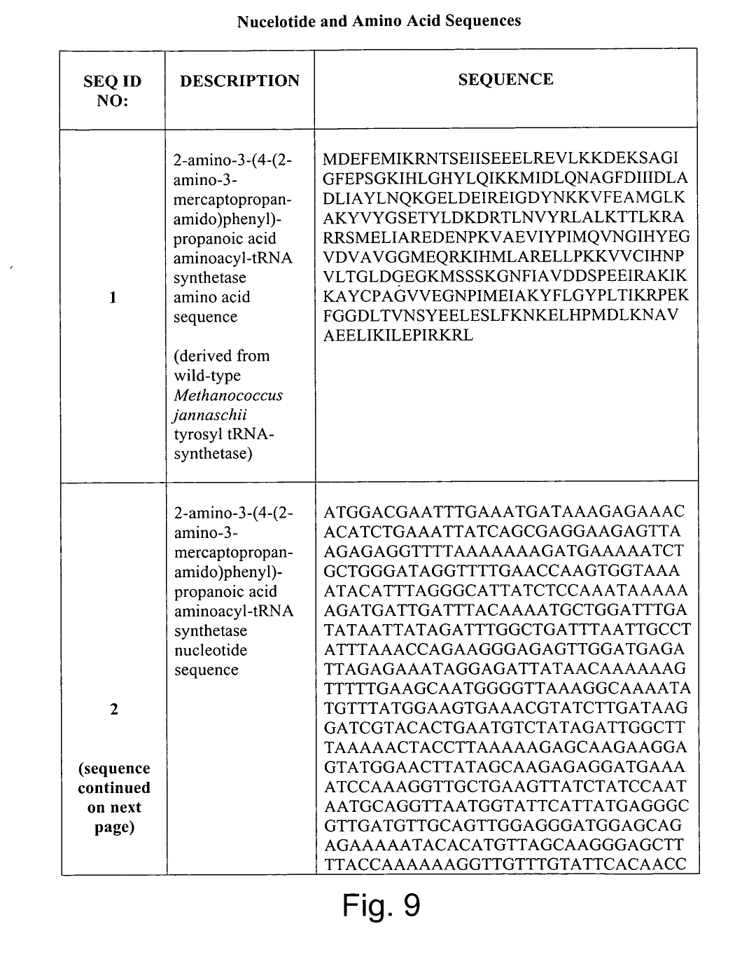

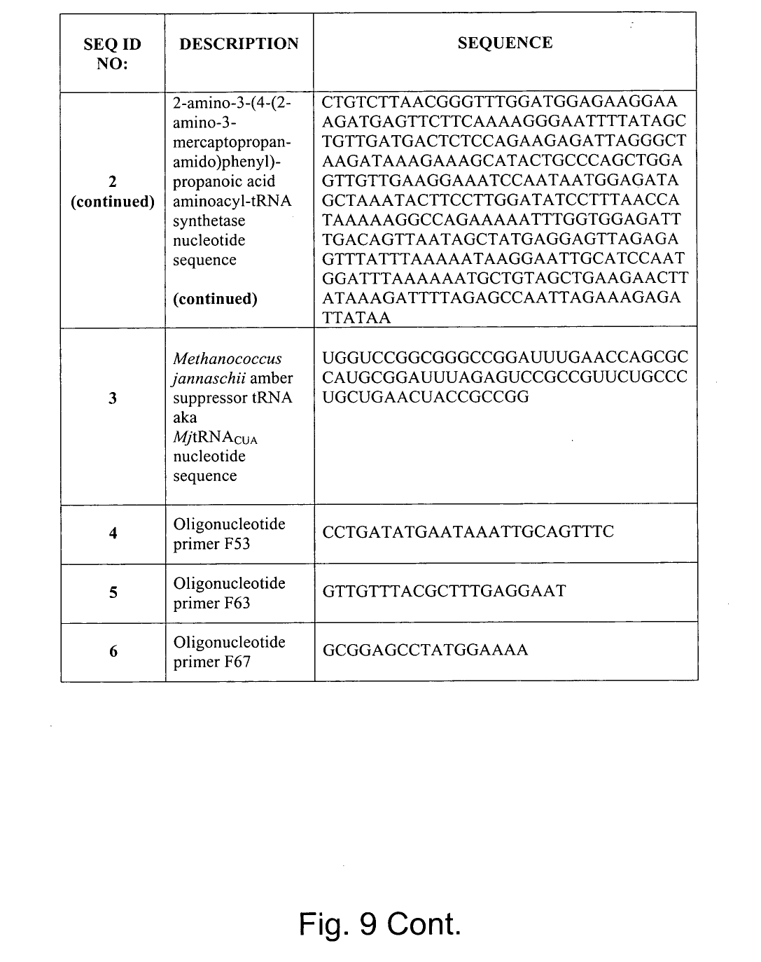

Examples of O-tRNAs of the invention are set forth in the sequence listing herein, for example, see FIG. 9 and SEQ ID NO: 3. The disclosure herein also provides guidance for the design of additional functionally similar O-tRNA species. In an RNA molecule, such as an O-RS mRNA, or O-tRNA molecule, Thymine (T) is replaced with Uracil (U) relative to a given sequence (or vice versa for a coding DNA), or complement thereof. Additional modifications to the bases can also be present to generate similar functionally equivalent molecules.

The invention also encompasses conservative variations of O-tRNAs corresponding to particular O-tRNAs herein. For example, conservative variations of O-tRNA include those molecules that function like the particular O-tRNAs, e.g., as in the sequence listing herein and that maintain the tRNA L-shaped structure by virtue of appropriate self-complementarity, but that do not have a sequence identical to that, e.g., in the sequence listing or FIG. 9, and desirably, are other than wild type tRNA molecules.

The composition comprising an O-tRNA can further include an orthogonal aminoacyl-tRNA synthetase (O-RS), where the O-RS preferentially aminoacylates the O-tRNA with an unnatural amino acid. In certain embodiments, a composition including an O-tRNA can further include a translation system, e.g., in vitro or in vivo. A nucleic acid that comprises a polynucleotide that encodes a polypeptide of interest, where the polynucleotide comprises a selector codon that is recognized by the O-tRNA, or a combination of one or more of these can also be present in the cell.

Methods for producing a recombinant orthogonal tRNA and screening its efficiency with respect to incorporating an unnatural amino acid into a polypeptide in response to a selector codon can be found, e.g., in International Application Publications WO 2002/086075, entitled “METHODS AND COMPOSITIONS FOR THE PRODUCTION OF ORTHOGONAL tRNA AMINOACYL-tRNA SYNTHETASE PAIRS;” WO 2004/094593, entitled “EXPANDING THE EUKARYOTIC GENETIC CODE;” and WO 2005/019415, filed Jul. 7, 2004. See also Forster, et al., (2003) “Programming peptidomimetic synthetases by translating genetic codes designed de novo.” Proc Natl Acad Sci USA 100: 6353-6357; and Feng, et al., (2003) “Expanding tRNA recognition of a tRNA synthetase by a single amino acid change.” Proc Natl Acad Sci USA 100: 5676-5681. Additional details are found in U.S. Pat. No. 7,045,337; U.S. Pat. No. 7,083,970; U.S. Pat. No. 7,238,510; U.S. Pat. No. 7,129,333; U.S. Pat. No. 7,262,040; U.S. Pat. No. 7,183,082; U.S. Pat. No. 7,199,222; and U.S. Pat. No. 7,217,809.

### Orthogonal Aminoacyl-tRNA Synthetase (O-RS)

The O-RS of the invention preferentially aminoacylates an O-tRNA with an unnatural amino acid that comprises a 1,2 aminothiol group, e.g., 2-amino-3-(4-(2-amino-3-mercaptopropanamido)phenyl)propanoic acid, in vitro or in vivo. The O-RS of the invention can be provided to the translation system, e.g., an E. coli cell, by a polypeptide that includes an O-RS and/or by a polynucleotide that encodes an O-RS or a portion thereof. For example, an example O-RS comprises an amino acid sequence as set forth in SEQ ID NO: 1, or a conservative variation thereof. In another example, an O-RS, or a portion thereof, is encoded by a polynucleotide sequence that encodes an amino acid comprising sequence in the sequence listing or examples herein, or a complementary polynucleotide sequence thereof. See, e.g., the polynucleotide of SEQ ID NO: 2.

General details for producing an O-RS, assaying its aminoacylation efficiency, and/or altering its substrate specificity can be found in Internal Publication Number WO 2002/086075, entitled “METHODS AND COMPOSITIONS FOR THE PRODUCTION OF ORTHOGONAL tRNA AMINOACYL-tRNA SYNTHETASE PAIRS;” and WO 2004/094593, entitled “EXPANDING THE EUKARYOTIC GENETIC CODE.” See also, Wang and Schultz “Expanding the Genetic Code,” Angewandte Chemie Int Ed 44: 34-66 (2005); and Hoben and Soll (1985) Methods Enzymol 113: 55-59, the contents of which are incorporated by reference in their entirety. Additional details are found in U.S. Pat. No. 7,045,337; U.S. Pat. No. 7,083,970; U.S. Pat. No. 7,238,510; U.S. Pat. No. 7,129,333; U.S. Pat. No. 7,262,040; U.S. Pat. No. 7,183,082; U.S. Pat. No. 7,199,222; and U.S. Pat. No. 7,217,809. Methods are also elaborated below.

Methods for identifying an orthogonal aminoacyl-tRNA synthetase (O-RS), e.g., an O-RS that preferentially aminoacylates a cognate O-tRNA with an unnatural amino acid that comprises a 1,2 aminothiol group, e.g., any of the unnatural amino acids shown in FIG. 1, are described in this disclosure (see the Example). For example, a method includes subjecting a population of cells to a positive and a negative selection. Each cell in the population comprises a member of a plurality of aminoacyl-tRNA synthetases (RSs). The plurality of RSs can include mutant RSs, RSs derived from a different species, e.g., a species other than that of the aforementioned cells, or both mutant RSs and RSs derived from a different species. Each cell in the population also comprises the orthogonal tRNA (O-tRNA), e.g., that can be derived from the same species as or a different species than that of the plurality of RSs. Each cell also comprises a polynucleotide that encodes a selection marker, e.g., a positive selection marker, and comprises at least one selector codon.

Cells are selected or screened for those that show an enhancement in suppression efficiency compared to cells lacking or comprising a reduced amount of the member of the plurality of RSs. Suppression efficiency can be measured by techniques known in the art and by techniques described in Xie, et al. (2005) “An expanding genetic code.” Methods 36: 227-238. Cells having an enhancement in suppression efficiency each comprise an active RS that can aminoacylate the O-tRNA with an unnatural amino acid comprising a 1,2 aminothiol group. The level of aminoacylation can be determined by a detectable substance, e.g., a labeled unnatural amino acid. An O-RS, identified by the method, is also a feature of the invention.

Any of a number of assays can be used to determine aminoacylation. These assays can be performed in vitro or in vivo. For example, in vitro aminoacylation assays are described in, e.g., Hoben and Soll (1985) Methods Enzymol. 113: 55-59. Aminoacylation can also be determined by using a reporter along with orthogonal translation components and detecting the reporter in a cell expressing a polynucleotide comprising at least one selector codon that encodes a protein. See also, WO 2002/085923, entitled “IN VIVO INCORPORATION OF UNNATURAL AMINO ACIDS;” and WO 2004/094593, entitled “EXPANDING THE EUKARYOTIC GENETIC CODE.”

Identified O-RSs, e.g., O-RSs capable of aminoacylating a cognate O-tRNA with an unnatural amino acid that comprises a 1,2 aminothiol group, can be further manipulated to alter their substrate specificities so that only a desired unnatural amino acid, but not any of the 20 natural, e.g., proteinogenic, amino acids are charged to the O-tRNA. Methods to generate an orthogonal aminoacyl-tRNA synthetase with a substrate specificity for an unnatural amino acid comprising a 1,2 aminothiol group include mutating the synthetase, e.g., at the active site in the synthetase, at the editing mechanism site in the synthetase, at different sites by combining different domains of synthetases, or the like, and applying a selection process in which a positive selection is followed by a negative selection. In the positive selection, suppression of the selector codon introduced at a nonessential position(s) of a positive marker allows cells to survive under positive selection pressure. In the presence of both natural and unnatural amino acids, survivors thus encode active synthetases charging the orthogonal suppressor tRNA with either a natural or unnatural amino acid, e.g., an unnatural amino acid comprising a 1,2 aminothiol group. In the negative selection, suppression of a selector codon introduced at a nonessential position(s) of a negative marker removes synthetases with specificities for natural amino acids. Survivors of the negative and positive selection encode synthetases that aminoacylate (charge) the orthogonal suppressor tRNA with unnatural amino acids only. These synthetases can then be subjected to further mutagenesis, e.g., DNA shuffling or other recursive mutagenesis methods, and iterative rounds of positive and negative selection.

A library of mutant O-RSs can be generated using various mutagenesis techniques known in the art. For example, the mutant RSs can be generated by site-specific mutations, random point mutations, homologous recombination, DNA shuffling or other recursive mutagenesis methods, chimeric construction or any combination thereof. For example, a library of mutant RSs can be produced from two or more other, e.g., smaller, less diverse “sub-libraries.” It should be noted that libraries of tRNA synthetases from various organism (e.g., microorganisms such as eubacteria or archaebacteria) such as libraries that comprise natural diversity (see, e.g., U.S. Pat. No. 6,238,884 to Short et al; U.S. Pat. No. 5,756,316 to Schallenberger et al; U.S. Pat. No. 5,783,431 to Petersen et al; U.S. Pat. No. 5,824,485 to Thompson et al; U.S. Pat. No. 5,958,672 to Short et al), are optionally constructed and screened for orthogonal pairs.

Once the synthetases are subject to the positive and negative selection/screening strategy, these synthetases can then be subjected to further mutagenesis. For example, a nucleic acid that encodes the O-RS can be isolated, and a set of polynucleotides that encode mutated O-RSs, e.g., generated by random mutagenesis, site-specific mutagenesis, recombination or any combination thereof, can be generated from the nucleic acid. These individual steps, or a combination of these steps, can be repeated until a mutated O-RS is obtained that preferentially aminoacylates the O-tRNA with an unnatural amino acid that comprises a 1,2 aminothiol group, e.g., any of the unnatural amino acids shown in FIG. 1. In some aspects of the invention, the steps are performed multiple times, e.g., at least two times.

Additional levels of selection/screening stringency can also be used in the methods of the invention, for producing O-tRNA, O-RS, or pairs thereof. The selection or screening stringency can be varied on one or both steps of the method to produce an O-RS. This could include, e.g., varying the amount of selection/screening agent that is used, etc. Additional rounds of positive and/or negative selections can also be performed. Selecting or screening can also comprise one or more of a change in amino acid permeability, a change in translation efficiency, a change in translational fidelity, etc. Typically, the one or more change is based upon a mutation in one or more gene in an organism in which an orthogonal tRNA-tRNA synthetase pair is used to produce protein.

### Source and Host Organisms

The orthogonal translational components (O-tRNA and O-RS) of the invention can be derived from any organism, or a combination of organisms, for use in a host translation system from any other species, with the caveat that the O-tRNA/O-RS components and the host system work in an orthogonal manner. It is not a requirement that the O-tRNA and the O-RS from an orthogonal pair be derived from the same organism. In some aspects, the orthogonal components are derived from archaebacterial genes for use in a eubacterial host system.

For example, the orthogonal O-tRNA can be derived from an archaebacterium, such as Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, Methanococcus maripaludis, Methanopyrus kandleri, Methanosarcina mazei (Mm), Pyrobaculum aerophilum, Pyrococcus abyssi, Sulfolobus solfataricus (Ss), Sulfolobus tokodaii, Thermoplasma acidophilum, Thermoplasma volcanium, or the like, or a eubacterium, such as Escherichia coli, Thermus thermophilus, Bacillus subtilis, Bacillus stearothermphilus, or the like, while the orthogonal O-RS can be derived from an organism or combination of organisms, e.g., an archaebacterium, such as Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, Methanococcus maripaludis, Methanopyrus kandleri, Methanosarcina mazei, Pyrobaculum aerophilum, Pyrococcus abyssi, Sulfolobus solfataricus, Sulfolobus tokodaii, Thermoplasma acidophilum, Thermoplasma volcanium, or the like, or a eubacterium, such as Escherichia coli, Thermus thermophilus, Bacillus subtilis, Bacillus stearothermphilus, or the like. In one embodiment, eukaryotic sources, e.g., plants, algae, protists, fungi, yeasts, animals, e.g., mammals, insects, arthropods, or the like can also be used as sources of O-tRNAs and O-RSs.

The individual components of an O-tRNA/O-RS pair can be derived from the same organism or different organisms. In one embodiment, the O-tRNA/O-RS pair is from the same organism. Alternatively, the O-tRNA and the O-RS of the O-tRNA/O-RS pair are from different organisms.

The O-tRNA, O-RS or O-tRNA/O-RS pair can be selected or screened in vivo or in vitro and/or used in a cell, e.g., a eubacterial cell, to produce a polypeptide with an unnatural amino acid. The eubacterial cell used is not limited, for example, Escherichia coli, Thermus thermophilus, Bacillus subtilis, Bacillus stearothermphilus, or the like. Compositions of eubacterial cells comprising translational components of the invention are also a feature of the invention.

See also, International Application Publication Number WO 2004/094593, entitled “EXPANDING THE EUKARYOTIC GENETIC CODE,” filed Apr. 16, 2004, for screening O-tRNA and/or O-RS in one species for use in another species. Additional details are found in Wang and Schultz, (2005) “Expanding the Genetic Code.” Angewandte Chemie Int Ed 44: 34-66; Xie and Schultz, (2005) “An Expanding Genetic Code.” Methods 36: 227-238; Xie and Schultz, (2005) “Adding Amino Acids to the Genetic Repertoire.” Curr Opinion in Chemical Biology 9: 548-554; and Wang et al., (2006) “Expanding the Genetic Code.” Annu Rev Biophys Biomol Struct 35: 225-249, and U.S. Pat. No. 7,045,337; U.S. Pat. No. 7,083,970; U.S. Pat. No. 7,238,510; U.S. Pat. No. 7,129,333; U.S. Pat. No. 7,262,040; U.S. Pat. No. 7,183,082; U.S. Pat. No. 7,199,222; and U.S. Pat. No. 7,217,809.

### Selector Codons

Selector codons of the invention expand the genetic codon framework of protein biosynthetic machinery. For example, a selector codon includes, e.g., a unique three base codon, a nonsense codon, such as a stop codon, e.g., an amber codon (UAG), or an opal codon (UGA), an unnatural codon, at least a four base codon, a rare codon, or the like. A number of selector codons can be introduced into a desired gene, e.g., one or more, two or more, more than three, etc. Conventional site-directed mutagenesis can be used to introduce the selector codon at the site of interest in a polynucleotide encoding a polypeptide of interest. See, e.g., Sayers, J. R., et al. (1988) “5′,3′ Exonuclease in phosphorothioate-based oligonucleotide-directed mutagenesis.” Nucl Acid Res 16: 791-802. By using different selector codons, multiple orthogonal tRNA/synthetase pairs can be used that allow the simultaneous site-specific incorporation of multiple unnatural amino acids e.g., including at least one unnatural amino acid, using these different selector codons.

Unnatural amino acids can also be encoded with rare codons. For example, when the arginine concentration in an in vitro protein synthesis reaction is reduced, the rare arginine codon, AGG, has proven to be efficient for insertion of Ala by a synthetic tRNA acylated with alanine. See, e.g., Ma, C. et al., (1993) “In vitro protein engineering using synthetic tRNA^{Ala} with different anticodons.” Biochemistry 32: 7939-7945. In this case, the synthetic tRNA competes with the naturally occurring tRNA^{Arg}, which exists as a minor species in Escherichia coli. In addition, some organisms do not use all triplet codons. An unassigned codon AGA in Micrococcus luteus has been utilized for insertion of amino acids in an in vitro transcription/translation extract. See, e.g., Kowal and Oliver, (1997) “Exploiting unassigned codons in Micrococcus luteus for tRNA-based amino acid mutagenesis.” Nucl Acid Res 25: 4685-4689. Components of the invention can be generated to use these rare codons in vivo.

Selector codons can also comprise extended codons, e.g., four or more base codons, such as, four, five, six or more base codons. Examples of four base codons include, e.g., AGGA, CUAG, UAGA, CCCU, and the like. Examples of five base codons include, e.g., AGGAC, CCCCU, CCCUC, CUAGA, CUACU, UAGGC and the like. Methods of the invention include using extended codons based on frameshift suppression. Four or more base codons can insert, e.g., one or multiple unnatural amino acids, into the same protein. In other embodiments, the anticodon loops can decode, e.g., at least a four-base codon, at least a five-base codon, or at least a six-base codon or more. Since there are 256 possible four-base codons, multiple unnatural amino acids can be encoded in the same cell using a four or more base codon. See also, Anderson, et al., (2002) “Exploring the Limits of Codon and Anticodon Size.” Chemistry and Biology 9: 237-244; Magliery, et al., (2001) “Expanding the Genetic Code: Selection of Efficient Suppressors of Four-base Codons and Identification of “Shifty” Four-base Codons with a Library Approach in Escherichia coli.” J Mol Biol 307: 755-769; Ma, C., et al., (1993) “In vitro protein engineering using synthetic tRNA^{Ala} with different anticodons.” Biochemistry 32:7939; Hohsaka, et al., (1999) “Efficient Incorporation of Nonnatural Amino Acids with Large Aromatic Groups into Streptavidin in In Vitro Protein Synthesizing Systems.” J Am Chem Soc 121: 34-40; and Moore, et al., (2000) “Quadruplet Codons: Implications for Code Expansion and the Specification of Translation Step Size.” J Mol Biol 298: 195-209. Four base codons have been used as selector codons in a variety of orthogonal systems. See, e.g., WO 2005/019415; WO 2005/007870 and WO 2005/07624. See also, Wang and Schultz, (2005) “Expanding the Genetic Code.” Angewandte Chemie Int Ed 44: 34-66, the content of which is incorporated by reference in its entirety.

For a given system, a selector codon can also include one of the natural three base codons, where the endogenous system does not use (or rarely uses) the natural base codon. For example, this includes a system that is lacking a tRNA that recognizes the natural three base codon, and/or a system where the three base codon is a rare codon.

Selector codons optionally include unnatural base pairs. Descriptions of unnatural base pairs that can be adapted for methods and compositions include, e.g., Hirao, et al., (2002) “An unnatural base pair for incorporating amino acid analogues into protein.” Nature Biotechnology 20: 177-182. See also Wu, et al., (2002) “Enzymatic Phosphorylation of Unnatural Nucleosides.” J Am Chem Soc 124: 14626-14630.

### Nucleic Acid and Polypeptide Sequences and Variants

As described herein, the invention provides for polynucleotide sequences encoding, e.g., O-tRNAs and O-RSs, and polypeptide amino acid sequences, e.g., O-RSs, and, e.g., compositions, systems and methods comprising said polynucleotide or polypeptide sequences. Examples of said sequences, e.g., O-tRNA and O-RS amino acid and nucleotide sequences are disclosed herein (see FIG. 9 and SEQ ID NOs: 1, 2, and 3). However, one of skill in the art will appreciate that the invention is not limited to those sequences disclosed herein, e.g., in the Examples and sequence listing. One of skill will appreciate that the invention also provides many related sequences with the functions described herein, e.g., polynucleotides and polypeptides encoding conservative variants of an O-RS disclosed herein.

Owing to the degeneracy of the genetic code, “silent substitutions”, i.e., substitutions in a nucleic acid sequence that do not result in an alteration in an encoded polypeptide, are an implied feature of every nucleic acid sequence that encodes an amino acid sequence. Similarly, “conservative amino acid substitutions,” where one or a limited number of amino acids in an amino acid sequence are substituted with different amino acids with highly similar properties, are also readily identified as being highly similar to a disclosed construct. Such conservative variations of each disclosed sequence are a feature of the present invention.

Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the polypeptide molecule. The following sets forth example groups that contain natural amino acids of like chemical properties, where substitutions within a group is a “conservative substitution”.

TABLE 1
Conservative Substitutions

| Nonpolar | | | | |
|---|---|---|---|---|
| and/or | | | | Negatively |
| Aliphatic | Polar, | | Positively | Charged |
| Side | Uncharged | Aromatic | Charged Side | Side |
| Chains | Side Chains | Side Chains | Chains | Chains |
| Glycine | Serine | Phenylalanine | Lysine | Aspartate |
| Alanine | Threonine | Tyrosine | Arginine | Glutamate |
| Valine | Cysteine | Tryptophan | Histidine | |
| Leucine | Methionine | | | |
| Isoleucine | Asparagine | | | |
| Proline | Glutamine | | | |

### Applications for Proteins and Polypeptides Comprising Unnatural Amino Acids That Comprise a 1,2 Aminothiol Group

Methods and compositions for producing genetically encoded polypeptides comprising at least one unnatural amino acid that comprises a 1,2 aminothiol group, e.g. any of the unnatural amino acids shown in FIG. 1, are a feature of this invention. The unnatural amino acids described herein can participate in native chemical ligation (NCL) reactions, in which a sulfhydryl group of the unnatural amino acid undergoes a transesterification step with an available thioester on a second moiety to form a thioester-linked intermediate which spontaneously rearranges, via an S- to N-acyl shift, to form a peptide bond. An unnatural amino acids that comprises a 1,2 aminothiol moiety can also be readily reacted with an aldehyde moiety to form a thiazolidine. Site-specific incorporation of unnatural amino acids comprising 1,2 aminothiol groups into expressed polypeptides expands the utility of NCL and/or of thiazolidine formation by varying the position and number of potential attachment sites in a polypeptide to which, e.g., a second amino acid in the polypeptide comprising the unnatural amino acid with a 1,2, aminothiol group, a second translationally synthesized polypeptide, a second synthetic peptide, a second semi-synthetic peptide, an oligonucleotide, a DNA, an RNA, a nucleotide analog, an affinity tag (e.g., biotin, FLAG, hexahistine, etc.), a synthetic drug, a carbohydrate derivative, a fluorophore (e.g., Cascade Blue, Alexa568, Alexa647, etc.), a chromophore (e.g., phytochrome, phycobilin, bilirubin, etc.), a spin label (such as nitroxide), a toxin, a metal chelator (such as nitrilotriacetate), a photocrosslinker (such as p-azidoiodoacetanilide), an NMR probe, an X-ray probe, a pH probe, an IR probe, a dye, a sugar, a hapten, a cofactor, a fatty acid, a terpene (e.g., geraniol, limonene, farnesol, etc.), a polyethylene glycol (e.g., a branched PEG, a linear PEG, PEGs of different molecular weights, etc.), a resin, a solid support, or the like, can be bound. The molecular entities comprising a thioester to which an unnatural amino acid comprising a 1,2 aminothiol group can be ligated via NCL, and/or the moieties comprising an aldehyde to which an unnatural amino acid comprising a 1,2 aminothiol group can be conjugated via a thiazolidine bond, are well known to those of skill in the art and are not particularly limiting, provided that they comprise a functional group that can participate in an NCL reaction with an aminothiol group, or react with an aldehyde group to form a thiazolidine. The expanded versatility of these reactions can be particularly useful in a myriad of applications in biotechnology, biomedical research, and chemical biology, including the study of protein structure, enzyme mechanism, protein-protein interactions, etc. It will be appreciated by those of skill in the art that the applications for polypeptides and proteins comprising an unnatural amino acid with a 1,2 aminothiol group that are described below are not to be taken as limiting.

Protein cyclization can provide insights into the importance of a protein's N- and C-termini in, e.g., protein folding and structural stability, can increase or prolong a protein's activity, and can protect a protein against proteolytic cleavage. Moreover, protein cyclization can reduce structural flexibility, which may aid the study or crystallization of proteins that are otherwise unstable in the absence of, e.g., a binding partner or a ligand. However, many of the synthetic methodologies developed for the cyclization of small bioactive peptides cannot be easily used in larger proteins. One solution to this problem can be to incorporate an unnatural amino acid that comprises a 1,2 aminothiol group, e.g., a 2-amino-3-(4-(2-amino-3-mercaptopropanamido)phenyl)propanoic acid, into a protein that is to be circularized. By incorporating a second unnatural amino acid comprising a thioester group or an aldehyde group into the same protein, the two functional groups can react, e.g., in vivo, via NCL or thiazolidine formation, respectively, to generate a cyclic peptide inside a living cell.

Interactions between proteins and other biomolecules, e.g., in live cells, can be captured in vivo using proteins that comprise unnatural amino acids that can participate in NCL reactions or form thiazolidine bonds, e.g., those UAA described herein. For example, a protein comprising such an unnatural amino acid, can be ligated in a site-specific manner via NCL, e.g., in vivo, to a crosslinker or photocrosslinker that comprises a thioester group, or via thiazolidine formation, e.g. to a crosslinker or photocrosslinker that comprises an aldehyde group. The modified protein can then be used to study the in vivo behavior of molecules in real time, capturing dynamic events, e.g., in the assembly and disassembly multiprotein complexes or in the activation or deactivation of signal transduction cascades.

Real-time protein folding, protein transport, and protein dynamics can also be monitored in vivo using proteins that comprise unnatural amino acids that can participate in NCL reactions or form thiazolidine bonds. A protein of interest comprising such an unnatural amino acid can be expressed in live cells that are then incubated with, e.g., a cell-permeable fluorescent probe (e.g., Cascade Blue, Alexa568, Alexa647, etc.), a dye, a pH probe, or a chromophore (e.g., phytochrome, bilirubin, phycobilin, etc.), that comprises a reactive thioester or aldehyde group. The probe can efficiently penetrate the cell membrane, and chemoselectively react with the 1,2 aminothiol group of the unnatural amino acid, producing a labeled protein of interest that can be monitored via, e.g., fluorescence microscopy.

Likewise, proteins comprising any one or more unnatural amino acids of the invention can be reacted with thioester or aldehyde-derivatized affinity tags (e.g., biotin, FLAG, hexahistidine, etc.) to facilitate, e.g., protein purification or imunnohistochemical analyses, such as western blots, ELISAs, antibody staining, etc.

The ability to conjugate polypeptides to nucleic acids, including RNA and DNA, is important in a number of life science applications. For example, a polypeptide-nucleic acid conjugate made using reacting, e.g., a fluorescent or a radioactive polypeptide comprising an amino acid depicted in FIG. 1 with, e.g., a thioester- or aldehyde-derivatized RNA, DNA, or oligonucleotide, can be useful in the labeling of nucleic acid probes for use in, e.g., Southern blots, northern blots, quantitative PCR, and other analyses.

Unnatural amino acids that comprise 1,2 aminothiol groups can be useful in studies in which NMR spectroscopy is used to determine a protein's structure. The incorporation of such an amino acid into a protein of interest allows the site-specific ligation of, e.g., a thioester- or aldehyde-bearing moiety comprising an NMR-sensitive nucleus, into, e.g., a discrete domain of a very large protein. This strategy can lead to simplified NMR spectra and can reduce the loss of spectral resolution that occurs as a result of increased line widths and increased numbers of signals with similar chemical shifts. Other aldehyde- or thioester-derivatized biophysical probes, e.g., spin labels (such as nitroxide), NMR probes, IR probes, X-ray probes, and the like, can be similarly conjugated to a protein of interest comprising, e.g., an unnatural amino acid depicted in FIG. 1, in preparation to investigate the protein's structural dynamics.

One of the most critical issues in the production of, e.g., a protein microarray is the development of protein immobilization methods. Optimally, a method would allow the efficient immobilization of proteins onto a solid support, e.g., a glass surface, while maintaining the proteins' native structures and biological functions. A target protein into which an unnatural amino acid comprising a 1,2 aminothiol group has been incorporated can be efficiently immobilized, e.g., via covalent bond formation, under aqueous conditions in a chemoselective manner, e.g., via NCL on a thioester-functionalized surface or via thiazolidine formation on an aldehyde-functionalized surface, e.g., a glass slide. The amino acid position at which the unnatural amino acid, e.g., an unnatural amino acid depicted in FIG. 1, is incorporated can be carefully chosen to insure that the activity and/or conformation of the target protein is not compromised by immobilization. A similar strategy can be used to immobilize a protein of interest onto a resin, a solid support.

Expressed protein ligation (EPL) is a protein semisynthesis method in which two or more, e.g., synthetic peptide segments or recombinantly expressed proteins, are covalently joined in a chemoselective manner via, e.g., NCL. This protein engineering technique can be used in to synthesize protein molecules, e.g., branched protein molecules, that possess altered structural and functional properties that would be useful tools in drug screening and in understanding complex processes such as, e.g., signal transduction and protein-ligand interactions. EPL can also be used to ligate, e.g., two or more folded protein domains to produce a desired chimera, which, if expressed translationally, would otherwise be unable to fold. However, EPL via NCL is still limited by the requirement that one of the peptide segments comprise an N-terminal Cys residue that can react with a thioester or aldehyde group in a second peptide segment. This constraint can be overcome by the translational incorporation of an unnatural amino acid that comprises a 1,2 aminothiol group, e.g., any of the unnatural amino acids described herein, into the peptide segment(s) that are to be ligated via NCL or via thiazolidine formation. Such an UAA can be incorporated into a polypeptide segment, e.g., a synthetic polypeptide segment, a semi-synthetic polypeptide segment, or a polypeptide segment produced via translation, at an amino acid position that permits the NCL reaction or thiazolidine bond formation without disturbing the segment's biological activity.

Glycosylation is one of the most common post-translational modifications of proteins in eukaryotes and affects a wide range of protein functions from folding and secretion to biomolecular recognition and serum half life. See, e.g., R. A. Dwek, (1996) “Glycobiology: toward understanding the function of sugars.” Chem Rev 96: 683-720. While there have been significant advances in our understanding of the effects of glycosylation, the specific roles of oligosaccharide chains and the relationships between their structures and functions are just beginning to be understood. See, e.g., C. R. Bertozzi, et al. (2001) “Chemical Glycobiology.” Science 291: 2357-2364. The primary challenge is that glycoproteins are typically produced as a mixture of glycoforms, making it difficult to isolate homogenous samples of, e.g., a glycoprotein of interest comprising a particular defined oligosaccharide structure, from natural sources. The translational incorporation of an unnatural amino acid that comprises a 1,2 aminothiol group, e.g., any of the unnatural amino acids described herein, into a protein of interest, followed by the attachment of an, e.g., carbohydrate or carbohydrate derivative of interest, to the desired protein at a defined amino acid position can permit the large-scale production of proteins that comprise a defined post-translational modification. This strategy can also be used to introduce a variety of additional modifications, e.g., a nucleotide analog, a metal chelator, a fatty acid, a terpene, a hapten, a toxin, a lipid, a PEG (e.g., a branched PEG, a linear PEG, PEGs of different molecular weights), or a synthetic drug (for a description of synthetic drugs, see, e.g., Remington: The Science and Practice of Pharmacy. University of the Sciences in Philadelphia, eds. (Lippincott Williams & Wilkins, 2006)), to a protein of interest in order to produce a homogenous sample.

A protein comprising an unnatural amino acid of the invention, e.g., an unnatural amino acid depicted in FIG. 1, to which a saccharide moiety has been ligated, e.g., via NCL, can be further glycosylated. Subsequent glycosylation steps can be carried out enzymatically, e.g., in vitro or in vivo, using, for example, a glycosyltransferase, glycosidase, or other enzyme known to those of skill in the art.

For enzymatic saccharide syntheses that involve glycosyltransferase reactions, the cells of the invention optionally contain at least one heterologous gene that encodes a glycosyltransferase. Many glycosyltransferases are known, as are their polynucleotide sequences. See, e.g., “The WWW Guide To Cloned Glycosyltransferases,” (available on the World Wide Web at www.vei.co.uk/TGN/gt_guide.htm). Glycosyltransferase amino acid sequences and nucleotide sequences encoding glycosyltransferases from which the amino acid sequences can be deduced are also found in various publicly available databases, including GenBank, Swiss-Prot, EMBL, and others.

Glycosyltransferases that can be employed, e.g., in the cells of the invention, e.g., to further modify saccharides ligated to proteins of interest at the site of an unnatural amino acid that comprises a 1,2 aminothiol group, include, but are not limited to, galactosyltransferases, fucosyltransferases, glucosyltransferases, N-acetylgalactosaminyltransferases, N-acetylglucosaminyltransferases, glucuronyltransferases, sialyltransferases, mannosyltransferases, glucuronic acid transferases, galacturonic acid transferases, oligosaccharyltransferases, and the like. Suitable glycosyltransferases include those obtained from eukaryotes, as well as from prokaryotes.

The glycosylation reactions which further modify sugars that have been ligated, e.g., via NCL, to a desired protein at the site of an unnatural amino acid comprising a 1,2 aminothiol group include, in addition to the appropriate glycosyltransferase and acceptor, an activated nucleotide sugar that acts as a sugar donor for the glycosyltransferase. The reactions can also include other ingredients that facilitate glycosyltransferase activity. These ingredients can include a divalent cation (e.g., Mg⁺² or Mn⁺²), materials necessary for ATP regeneration, phosphate ions, and organic solvents. The concentrations or amounts of the various reactants used in the processes depend upon numerous factors including reaction conditions such as temperature and pH value, and the choice and amount of acceptor saccharides to be glycosylated. The reaction medium may also comprise solubilizing detergents (e.g., Triton or SDS) and organic solvents such as methanol or ethanol, if necessary.

Further details regarding systems and methods for the preparation of glycoproteins using proteins that comprise unnatural amino acids are elaborated in U.S. patent application Ser. No. 11/255,601, titled “IN VIVO SITE-SPECIFIC INCORPORATION OF N-ACETYL-GALACTOSAMINE AMINO ACIDS IN EUBACTERIA” and U.S. Pat. No. 6,927,042, titled “GLYCOPROTEIN SYNTHESIS”, the contents of each of which are incorporated by reference in their entirety.

### Proteins and Polypeptides of Interest

Essentially any protein (or portion thereof) that includes an unnatural amino acid that comprises a 1,2 aminothiol group, e.g., an unnatural amino acid shown in FIG. 1, can be produced using the compositions and methods herein. No attempt is made to identify the hundreds of thousands of known proteins, any of which can be modified to include one or more unnatural amino acids that comprise a 1,2 aminothiol group, e.g., by tailoring any available mutation methods to include one or more appropriate selector codons in a relevant translation system. Common sequence repositories for known proteins include GenBank EMBL, DDBJ and the NCBI. Other repositories can easily be identified by searching the internet.

Typically, the proteins are, e.g., at least 60%, at least 70%, at least 75%, at least 80%, at least 90%, at least 95%, or at least 99% or more identical to any available protein (e.g., a therapeutic protein, a diagnostic protein, an industrial enzyme, or portion thereof, and the like), and they comprise one or more unnatural amino acids of the invention. In some embodiments, polypeptides that can comprise at least one unnatural amino acid that comprises a 1,2 aminothiol group include a Z domain of Staphylococcal protein A, an SH3 domain, and c-Crk.

A classical “Z domain” is a 58-residue three-helix bundle derived from an IgG Fc-binding domain of Staphylococcus aureus Protein A. Randomization and substitution of the surface-exposed amino acids comprising this scaffold protein, e.g., with an unnatural amino acid comprising a 1,2 aminothiol group, can be useful in generating affinity ligands capable of binding desired target proteins. Affinity ligands produced in this manner can become widespread tools in a variety of biotechnological and biomedical applications, e.g., affinity purification (Lamla, et al. (2004) “The Nano-tag, a streptavidin-binding peptide for the purification and detection of recombinant proteins.” Protein Expr Purif 33: 39-47), protein microarray technology (Renberg et al. (2005) “Affibody protein capture microarrays: Synthesis and evaluation of random and directed immobilization of affibody molecules.” Anal Biochem 341: 334-343), bioimaging (Wikman et al. (2004) “Selection and characterization of HER2/neu-binding affibody ligands.” Protein Eng Des Sel 17: 455-462), enzyme inhibition (Amstutz et al. (2005) “Intracellular kinase inhibitors selected from combinatorial libraries of designed ankyrin repeat proteins.” J Biol Chem 280: 24715-24722), and potential targeted drug delivery (Heyd et al. (2003) “In vitro evolution of the binding specificity of neocarzinostatin, an enediyne-binding chromoprotein.” Biochemistry 42: 5674-5683; Nicaise et al. (2004) “Affinity transfer by CDR grafting on a nonimmunoglobulin scaffold.” Protein Sci 13: 1882-1891).

Src homology 3 (SH3) domains are non-catalytic protein modules that have been identified in hundreds of signaling proteins in diverse eukaryotic species ranging from yeast to human (Mayer (2001) “SH3 domains: complexity in moderation.” Journal Cell Sci 114: 1253-1263; Tong, et al. (2002) “A combined experimental and computational strategy to define protein interaction networks for peptide recognition modules.” Science 295: 321-324). Members of the SH3 family, which typically comprise 50-70 amino acid residues, recognize specific proline-rich sequence motifs in target proteins and act as molecular adhesives that mediate protein-protein interactions in a variety of biological processes. SH3 domains play critical roles in the formation of multiprotein complexes, the formation molecular networks responsible for signal transduction, and in the regulation of enzyme activity and cytoskeletal organization (reviewed in Morton, et al. (1994) “SH3 domains: Molecular Velcro.” Curr Biol 4: 615-617; Mayer (2001) “SH3 domains: complexity in moderation.” Journal Cell Sci 114: 1253-1263). The versatile nature of SH3 domains in target protein recognition suggests that these modular domains are adaptable and, like Z domain, can also find use as affinity reagents that can be tailored to generate ligands that possess prescribed specificities. Substitution of amino acids in an SH3 domain with, e.g., an unnatural amino acid comprising a 1,2 aminothiol group, can be useful in tailoring such affinity ligands.

c-Crk is a member of a family of signaling proteins whose modular domain architecture consists largely of an Src homology 2 domain (SH2) followed by two SH3 domains. Crk family proteins are widely expressed and mediate the formation of signal transduction protein complexes in response to a variety of extracellular stimuli, including growth and differentiation factors (reviewed in Feller (2001) “Crk family adaptors-signaling complex formation and biological roles.” Oncogene 20: 6348-71). The upstream signaling partners which selectively bind to c-Crk include various receptors (Sorokin, et al. (1998) “Crk protein binds to PDGF receptor and insulin receptor substrate-1 with different modulating effects on PDGF- and insulin-dependent signaling pathways.” Oncogene 16: 2425-2434; Furge, et al. (2000) “Met receptor tyrosine kinase: enhanced signaling through adapter proteins.” Oncogene 19: 5582-9) and large multisite docking proteins (Feller, et al. (2006) “Potential disease targets for drugs that disrupt protein-protein interactions of Grb2 and Crk family adaptors.” Curr Pharm Des 12: 529-548; Huang, et al. (2006) “The docking protein Cas links tyrosine phosphorylation signaling to elongation of cerebellar granule cell axons.” Mol Bio Cell 17: 3187-3196), while several protein kinases and guanine nucleotide release proteins (GNRPs) have been suggested to function downstream of c-Crk to effect, e.g., cell motility, adhesion, and cell growth regulation (Tanaka, et al. (1994) “C3G, a Guanine Nucleotide-Releasing Protein Expressed Ubiquitously, Binds to the Src Homology 3 Domains of CRK and GRB2/ASH Proteins.” Proc Natl Acad Sci USA 91: 3443-3447; Polte, et al. (1997) “Complexes of Focal Adhesion Kinase (FAK) and Crk-associated Substrate (p130^{Cas}) Are Elevated in Cytoskeleton-associated Fractions following Adhesion and Src Transformation.” Journal Biol Chem 272: 5501-5509, reviewed in Feller (2001) “Crk family adaptors-signaling complex formation and biological roles.” Oncogene 20: 6348-71). The SH2 domains of c-Crk can interact with upstream signaling molecules (Anafi, et al. (1997) “SH2/SH3 Adaptor Proteins Can Link Tyrosine Kinases to a Ste20-related Protein Kinase, HPK1.” J Biol Chem 44: 27804-27811), whereas the SH3 domains of c-Crk are critical for the coupling of this adaptor protein to effector molecules and for the targeting of signaling complexes to discrete sites within the cell (Bar-Sagi, et al. (1993) “SH3 domains direct cellular localization of signaling molecules.” Cell 74: 83-91). Substitution of amino acids in the SH2 and SH3 domains of Crk with, e.g., a UAA comprising a 1,2 aminothiol group, can produce Crk variants with altered target protein specificities and/or Crk variants that respond to altered upstream stimuli.

Other examples of therapeutic, diagnostic, and other polypeptides that can comprise at least one unnatural amino acid that comprises a 1,2 aminothiol group, e.g., a 2-amino-3-(4-(2-amino-3-mercaptopropanamido)phenyl)propanoic acid residue, include, but are not limited to, those in International Publications WO 2004/094593, filed Apr. 16, 2004, entitled “Expanding the Eukaryotic Genetic Code;” and, WO 2002/085923, entitled “IN VIVO INCORPORATION OF UNNATURAL AMINO ACIDS.” Such proteins and polypeptide domains include aldosterone receptor, alpha-1 antitrypsin, angiostatin, antibody fragments, antihemolytic factor, apolipoprotein, apoprotein, atrial natriuretic factor, an atrial peptide, calcitonin, CC chemokine, CD40, CD40 ligand, CD44, collagen, colony stimulating factor (CSF), complement factor 5a, complement inhibitor, complement receptor 1, corticosterone, C—X—C chemokine, a cytokine, D31065, DHFR, ENA-78, estrogen receptor, epidermal growth factor (EGF), an epithelial neutrophil activating peptide, epithelial Neutrophil Activating Peptide-78, erythropoietin (EPO), exfoliating toxin, Factor VII, Factor VIII, Factor IX, Factor X, fibrinogen, fibroblast growth factor (FGF), fibronectin, Fos, GCP-2, G-CSF, glucocerebrosidase, GM-CSF, gonadotropin, Gro-a, Gro-b, Gro-c, GROα, GROβ, GROγ, a growth factor, a growth factor receptor, HCC1, a hedgehog protein, hemoglobin, hepatocyte growth factor (HGF), human serum albumin (HAS), human growth hormone (HGH), hyalurin, I309, ICAM-1, ICAM-1 receptor, IFN-α, IFN-β, IFN-γ, IGF-I, IGF-II, IL-1, IL-2, IL-3, IL-4, IL-5, IL-6, IL-7, IL-8, IL-9, IL-10, IL-11, IL-12, inflammatory molecules, insulin, insulin-like growth factor (IGF), an interferon, an interleukin, IP-10, Jun, keratinocyte growth factor (KGF), Lactoferrin, LDL receptor, leukemia inhibitory factor, LFA-1, LFA-1 receptor, luciferase, MCP-1, Met, MGSA, MIG, MIP1-α, MIP1-β, MIP1-δ, monocyte chemoattractant protein-1, monocyte chemoattractant protein-2, monocyte chemoattractant protein-3, Mos, Myb, Myc, NAP-2, NAP-4, Neurturin, neutrophil inhibitory factor (NIF), an oncogene product, oncostatin M, osteogenic protein-1, p53, parathyroid hormone, PD-ECSF, PDGF, a peptide hormone, PF4, pleiotropin, progesterone receptor, Protein A, Protein G, pyrogenic exotoxins A, B, or C, R83915, R91733, Raf, RANTES, Ras, Rel, relaxin, renin, SCF/c-kit, SDF-1, SEA, SEB, SEC1, SEC2, SEC3, SED, SEE, a signal ransduction molecule, soluble complement receptor I, soluble I-CAM 1, soluble interleukin receptor, soluble TNF receptor, somatomedin, somatostatin, a somatotropin, a Staphylococcal enterotoxin, a steroid hormone receptor, streptokinase, a superantigen, superoxide dismutase (SOD), T39765, T58847, T64262, Tat, a testosterone receptor, TGF-α, TGF-β, thymosin alpha 1, tissue plasminogen activator, Toxic Shock Syndrome toxin, transcriptional activators, transcriptional repressors, a tumor growth factor (TGF), tumor necrosis factor (TNF), TNF alpha, TNF beta, a tumor necrosis factor receptor (TNFR), urokinase, vascular endothelial growth factor (VEGEF), VCAM-1 protein, and VLA-4 protein.

### EXAMPLES

The following examples are offered to illustrate, but not to limit the claimed invention. It is understood that the examples and embodiments described herein are for illustrative purposes only and that various modifications or changes in light thereof will be suggested to persons skilled in the art and are to be included within the spirit and purview of this application and scope of the appended claims.

### SYNTHESIS OF 2-AMINO-3-(4-(2-AMINO-3-MERCAPTOPROPANAMIDO)PHENYL)PROPANOIC ACID

The materials used in the synthesis of 2-amino-3-(4-(2-amino-3-mercaptopropanamido)phenyl)propanoic acid were obtained from the following sources: Water was taken from a Milli-Q ultra pure water purification system (Millipore). Peptide synthesis-grade dimethylformamide (DMF) was purchased from Alfa Aesar. N-(tert butoxycorbonyl)-4-aminophenylalanine (Boc-p-amino-Phe-OH) was purchased from Bachem. N-(tert butoxycarbonyl)-S-(triphenylmethyl)cysteine (Boc-Cys(Trt)-OH) and 1-hydroxybenzotriazole hydrate (HOBt) were purchased from Novabiochem. HPLC-grade acetonitrile was purchased from Fisher Scientific. Deuterated solvents, which were used to solubilize synthesized compounds in preparation for NMR characterization, were purchased from Cambridge Isotope Laboratories Inc. Other commercial reagents were purchased from Acros Organics and were used without further purification.

High-resolution mass spectra were measured on an Agilent 6210 Time of Flight Mass Spectrometer. Preparative HPLC was run on a Hitachi (D-7000 HPLC system) instrument using a preparative column (Grace Vydac “Protein & Peptide C18”, 250×22 mm, 10-15 μm particle size, flow rate 8 mL/min). Detection of the signal was achieved with either photodiode array or UV detector at a wavelength of λ=260 nm. Eluents A (0.1% trifluoroacetic acid (TFA) in water) and B (0.1% TFA in acetonitrile) were used in a linear gradient.

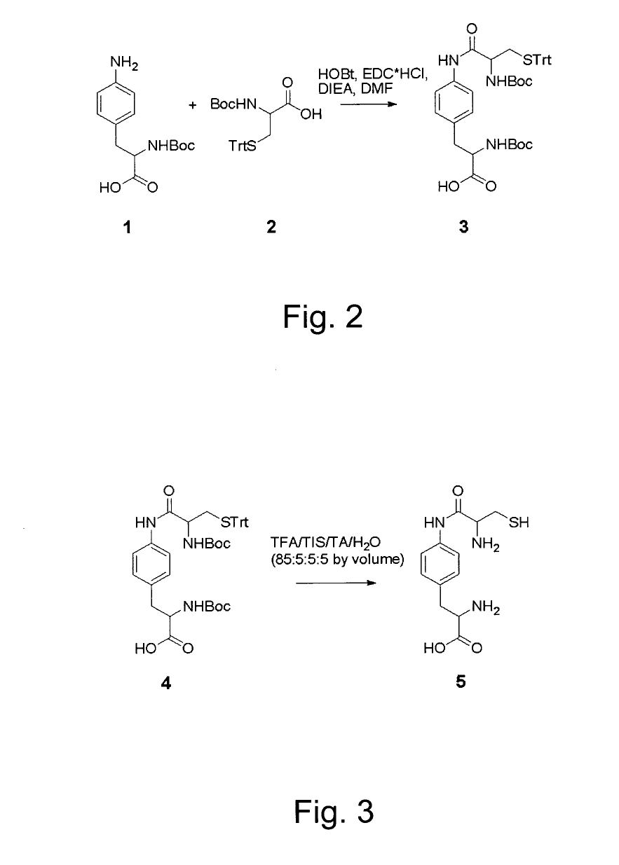

Two reaction schemes were followed to synthesize 2-amino-3-(4-(2-amino-3-mercaptopropanamido)phenyl)propanoic acid. In the first reaction scheme, shown in FIG. 2, Compound 1 Boc-Cys(Trt)-OH (1.6 g, 3.53 mmol), 1-(3-dimethylaminopropyl)-3-ethylcarbodiimide hydrochloride (EDC*HCl) (600 mg, 3.9 mmol) and HOBt (500 mg, 3.9 mmol) were dissolved in anhydrous DMF (35 mL). Trt=trityl. N,N-diisopropylethylamine (DIEA) (668 μL, 3.9 mmol) was added and the reaction mixture was stirred under argon for 5 min, followed by the addition of Compound 2 Boc-p-amino-Phe-OH (900 mg, 3.21 mmol). After stirring for 12 h, the solvent was removed from the solution via vacuum, and the residue was purified by column chromatography (eluent: first 3:1 v/v hexane/ethyl acetate, then 2:1 v/v hexane/ethyl acetate) to afford Compound 3 2-(tert-butoxycarbonylamino)-3-(4-(2-(tert-butoxycarbonylamino)-3-(tritylthio)propanamido)phenyl)propanoic acid as a white solid which was directly used in the next synthesis step (R_{F} (eluent: 2:1 v/v hexane/ethyl acetate)=0.13).

In the second reaction scheme, shown in FIG. 3, Compound 3 Boc-Cys(Trt)-OH (1.6 g, 3.53 mmol) was dissolved in a mixture of TFA (trifluoroacetic acid), triisopropylsilane (TIS), thioanisole (TA) and water (50 mL, 85:5:5:5 by volume) and stirred for 20 min. The solvent was removed from the solution via vacuum, and the residue was purified by preparative reversed phase HPLC to afford, after lyophylisation, Compound 5 as a white solid. NMR spectra of Compound 5 are as follows: ¹H NMR (500 MHz, MeOH-d₄): δ 3.05 (m, 2H, CH₂), 3.15 (m, 2H, CH₂), 4.17 (m, 1H, HSCH₂C(NH₂)H), 4.22 (m, 1H, HCNH₂COOH), 7.29 (d, J=8.5 Hz, 2H, 2×aromatic H), 7.62 (d, J=8.5 Hz, 2H, 2 aromatic H); ¹³C NMR (125 MHz, MeOH-d₄): δ 26.4, 36.9, 55.2, 56.8, 121.9, 131.2, 132.2, 138.6, 166.8, 171.3. These results indicate that Compound 5 possesses the chemical structure of the unnatural amino acid 2-amino-3-(4-(2-amino-3-mercaptopropanamido)phenyl)propanoic acid. The yield of Compound 5 is 23% over the two synthesis steps.

The results of high-resolution mass spectrometry (electrospray sample ionization-time of flight), or HRMS (ESI-TOF), analysis of Compound 5 are as follows: m/z calculated for C₁₂H₁₇N₃O₃S [M+H]⁺: 284.1063; found: 284.1069, indicating that the synthesized compound exhibited the expected molecular mass.

### SELECTION FOR A M. JANNASCHII-DERIVED ORTHOGONAL AMINOACYL-tRNA SYNTHETASE (O-RS) SPECIFIC FOR THE UNNATURAL AMINO ACID 2-AMINO-3-(4-(2-AMINO-3-MERCAPTOPROPANAMIDO)PHENYL)PROPANOIC ACID

Based on an analysis of the X-ray crystal structure of the Methanococcus Jannaschii tyrosine synthetase with its cognate amino acid or aminoacyl adenylate (Kobayashi, et al. (2003) “Structural basis for orthogonal tRNA specificities of tyrosyl-tRNA synthetases for genetic code expansion.” Nature Struct Biol 10: 425-432; Brick, et al. (1989) “Structure of tyrosyl-tRNA synthetase refined at 2.3 Å resolution interaction of the enzyme with the tyrosyl adenylate intermediate.” J Mol Biol 208: 83-98), a library of synthetase variants was generated by randomizing 10 amino acid residues: Tyr32 to codon RGK (Gly, Arg or Ser), Leu65 to codon NNT (Ala, Arg, Asn, Asp, Cys, Gly, His, Ile, Leu, Phe, Pro, Ser, Thr, Tyr or Val), Ala67 to codon GST (Ala or Gly), His70 to codon NNT, Phe108 to codon NNK (all amino acids), Gln109 to codon NNK, Tyr114 to codon VNT (Ala, Arg, Asn, Asp, Gly, His, Ile, Leu, Pro, Ser, Thr or Val), Asp158 to codon RST (Ala, Gly, Ser or Thr), Leu162 to codon VVK (Ala, Arg, Asn, Asp, Gln, Glu, Gly, His, Lys, Pro, Ser or Thr) and Ile159 to codon NNT. This library was constructed by overlap extension polymerase chain reaction (PCR) using synthetic degenerate oligonucleotide primers to introduce the mutations described above. These methods are described in more detail in, e.g., Xie, et al. (2005) “An expanding genetic code.” Methods 36: 227-238. The PCR-derived synthetase variants were cloned into the pBK plasmid and transformed into E. coli strain DH10B. The theoretical diversity of this library is 2.9×10¹⁰, based on codon diversity, and 4.7×10⁹, based on amino acid diversity. In practice, a library diversity of 1×10¹⁰ was achieved.

Selections for an orthogonal synthetase (O-RS) capable of charging its cognate orthogonal tRNA (O-tRNA) with 2-amino-3-(4-(2-amino-3-mercaptopropanamido)phenyl)propanoic acid were performed as previously described in, e.g., Xie, et al. (2005) “An expanding genetic code.” Methods 36: 227-238. Colonies were selected on LB agar plates that contained 1 mM 2-amino-3-(4-(2-amino-3-mercaptopropanamido)phenyl)propanoic acid. The plates were prepared with a 100 mM stock solution of the unnatural amino acid that was filtered through a 0.2 μM syringe and stored at −20° C. (2-amino-3-(4-(2-amino-3-mercaptopropanamido)phenyl)propanoic acid is a white powder that dissolves well in water.) After 3 rounds of positive selection that were alternated with 2 rounds of negative selection, 4×96 colonies were picked and spotted onto 3 sets LB plates containing 20, 40, or 50 μg/ml chloramphenicol; and onto 3 sets of LB plates containing 1 mM 2-amino-3-(4-(2-amino-3-mercaptopropanamido)phenyl)propanoic acid and 50, 66 or 110 μg/ml chloramphenicol. These spottings were done to estimate growth. Only one clone exhibited the desired phenotype, e.g., chloramphenicol resistance and growth only on media supplemented with 2-amino-3-(4-(2-amino-3-mercaptopropanamido)phenyl)propanoic acid (plate 2, position H8). The clone grew well on an LB agar plate containing 1 mM of the unnatural amino acid and 66 μg/ml chloramphenicol and exhibited fair growth on LB agar with 1 mM of the unnatural amino acid and 110 μg/ml chloramphenicol. The clone did not grow on plates in the absence of 1 mM 2-amino-3-(4-(2-amino-3-mercaptopropanamido)phenyl)propanoic acid. The plasmid encoding the putative desired O-RS was isolated and sequenced with the following primers: F53, e.g., SEQ ID NO: 4, CCTGATATGAATAAATTGCAGTTTC; F63, e.g., SEQ ID NO: 5, GTTGTTTACGCTTTGAGGAAT; and F67, e.g., SEQ ID NO: 6, GCGGAGCCTATGGAAAA. The MjTyrRS mutant that was obtained has the mutations shown in Table 2 below.

TABLE 2

| | WT RS | WT RS | Mutant | Mutant RS |
|---|---|---|---|---|
| Amino Acid Position | codon | amino acid | RS codon | amino acid |
| 32 | TAC | Tyr | GGG | Gly |
| 65 | TTG | Leu | GAT | Asp |
| 67 | GCT | Ala | GCT | Ala |
| | | | | (silent mutation) |
| 70 | CAC | His | ATT | Ile |
| 76 (this position was not randomized | AAA | Lys | AAG | Lys |
| in the library; it is posited to be a | | | | (silent mutation) |
| spontaneous mutation) | | | | |
| 84 (this position was not randomized | AAA | Lys | GAA | Glu |
| in the library; it is posited to be a | | | | |
| spontaneous mutation) | | | | |
| 108 | TTC | Phe | ACG | Thr |
| 109 | CAG | Gln | TAT | Tyr |
| 114 | TAT | Tyr | CGT | Arg |
| 158 | GAT | Asp | GGT | Gly |
| 159 (this position was not randomized | ATT | Ile | ATT | Ile |
| in the library; it is posited to be a | | | | (silent) |
| spontaneous mutation) | | | | |
| 162 | TTA | Leu | GAG | Glu |
| 250 (this position was not randomized | GAA | Glu | GGA | Gly |
| in the library; it is posited to be a | | | | |
| spontaneous mutation) | | | | |

### VERIFYING THE SPECIFICITY AND FIDELITY OF THE INCORPORATION OF 2-AMINO-3-(4-(2-AMINO-3-MERCAPTOPROPANAMIDO)PHENYL)PROPANOIC ACID INTO Z-DOMAIN PROTEIN IN E. COLI BY THE MUTANT M. JANNASCHII 2-AMINO-3-(4-(2-AMINO-3-MERCAPTOPROPANAMIDO)PHENYL)PROPANOIC ACID TRNA SYNTHETASE (RS)

Experiments to determine the efficiency and fidelity of incorporation of 2-amino-3-(4-(2-amino-3-mercaptopropanamido)phenyl)propanoic acid into proteins were performed as previously described (Xie, et al. (2004) “The site-specific incorporation of p-iodophenylalanine for structure determination.” Nature Biotech 22: 1297-1301). Plasmid pBK, which encodes the mutant M. jannaschii 2-amino-3-(4-(2-amino-3-mercaptopropanamido)phenyl)propanoic acid tRNA synthetase (RS), and plasmid pLeiZ, which encodes a C-terminal 6 His tagged mutant Z-domain protein with an amber codon at amino acid position 7 and the mutRNA_{CUA}^{Tyr}, were cotransformed into both competent E. coli DH10B cells and competent E. coli GeneHog (Invitrogen) cells. The transformed cells were recovered in SOC for 1 hour prior to plating on LB again plates containing 50 μg/mL kanamycin (to select for pBK transformants) and 34 μg/ml chloramphenicol (to select for pLeiZ transformants). The plates were then incubated at 37° C. for 14-18 hours.

A single kanamycin^{R} chloramphenicol^{R} colony was picked from the plates and used to inoculate 6 mL 2YT medium containing 50 μg/mL kanamycin and 34 μg/ml chloramphenicol. When the culture was grown to near saturation, 500 μL of this culture were used to inoculate 15 mLs of GMML medium containing 50 μg/mL kanamycin and 34 μg/ml chloramphenicol. GMML medium is a glycerol minimal medium comprising leucine (1×M9 salts (Sigma), 1 mM MgSO₄, 0.1 mM CaCl₂, 0.5 g/L NaCl, 0.3 mM L-leucine, 1% (vol/vol) glycerol). When the GMML culture was grown to near saturation, 8 mLs of this culture were used to inoculate 200 mLs of GMML medium containing 50 μg/mL kanamycin, 34 μg/ml chloramphenicol, and 1.0 mM 2-amino-3-(4-(2-amino-3-mercaptopropanamido)phenyl)propanoic acid. A second culture of GMML medium containing 50 μg/mL kanamycin, 34 μg/ml chloramphenicol and no UAA was also inoculated and grown in parallel. When these 200 mL cultures reached an OD₆₀₀ of approximately 0.5-0.6, isopropyl-β-D-thiogalactopyranoside (IPTG) was added to a final concentration of 1 mM to induce expression of Z-domain protein. After 4 hours, the cells were pelleted via centrifugation.

The cell pellets were resuspended in 6 mLs of Buffer B (100 mM NaH₂PO₄, 10 mM Tris-HCl, 8M Urea, pH=8.0) and lysed via sonication (3×30 second sonication cycles followed by 60 second incubations on ice). The sonicated whole cell lysates were centrifuged at 10,000 g for 30 minutes at 25° C., and the recovered supernatants were mixed with 1 mL of 50% Ni-NTA agarose slurry. The supernatant/slurry mixtures were shaken gently at room temperature for 60 minutes and then each loaded onto a column (available from Qiagen). The column flow-through was collected, and the columns were then washed twice with 4 mLs of Buffer C (100 mM NaH₂PO₄, 10 mM Tris-HCl, 8M urea, pH=6.8, buffer prepared right before use), and the recombinant Z-domain protein was eluted from each column with 5×0.5 mL Buffer E (100 mM NaH₂PO₄, 10 mM Tris-HCl, 8M urea, pH=4.5, buffer prepared right before use). All washes and elutions were also collected.

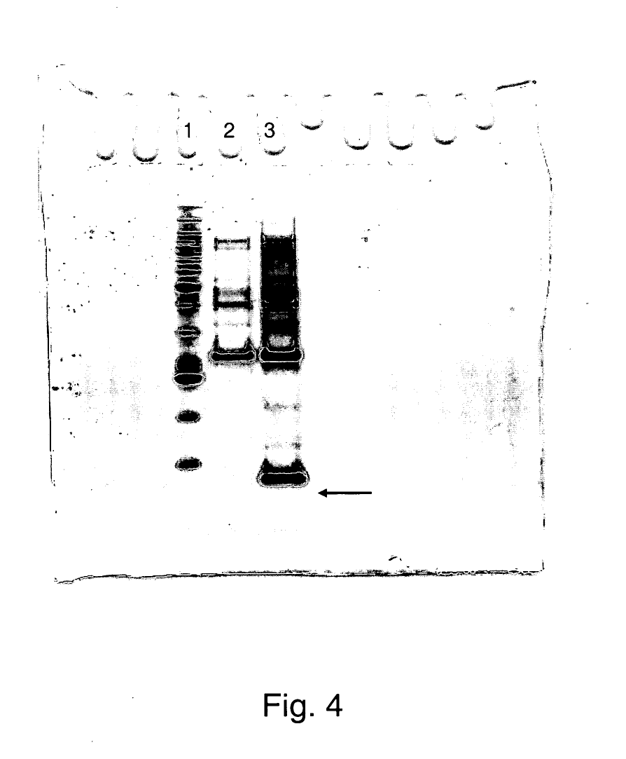

The eluted fractions, column washes, and flow-through were assayed by SDS PAGE (10-20% polyacrylamide, or 15% Tris Glycine PAGE), and the gel was then stained with GelCode (Pierce) or silver stain to visualize the results of the Z-domain protein expression and purification experiments described above. A GelCode-stained SDS PAGE on which purified protein samples derived from cultures grown in the absence (lane 2) or presence (lane 3) 2-amino-3-(4-(2-amino-3-mercaptopropanamido)phenyl)propanoic acid is shown in FIG. 4. Molecular weight markers (Benchmark™, available from Invitrogen) were run in lane 1. FIG. 4 shows that full-length Z-domain protein (indicated with an arrow) was expressed only in cells cultured in media to which 2-amino-3-(4-(2-amino-3-mercaptopropanamido)phenyl)propanoic acid was added. (FIG. 4, lane 3). Results of ESI-MS analysis show the other major band in FIG. 4, lanes 2 and 3 is most likely an E. coli petidyl-prolyl-cis-trans-isomerase, an E. coli protein comprising a long sequence of histidine residues. These results indicate that the 2-amino-3-(4-(2-amino-3-mercaptopropanamido)phenyl)propanoic acid RS recognizes this UAA with high specificity and does not recognize endogenous amino acids in E. coli to any significant degree.

Electrospray ionization mass spectrometry (ESI-MS) analysis of both the purified UAA and wild type Z-domain proteins was performed at the Genomics Institute of the Novartis Foundation (GNF) to confirm the incorporation of 2-amino-3-(4-(2-amino-3-mercaptopropanamido)phenyl)propanoic acid into the UAA Z-domain mutant. The difference in mass between the wt Z-domain protein, which comprises a tyrosine residue (molecular mass=181.07 Da) at amino acid position 7, and UAA Z-domain, which comprises a 2-amino-3-(4-(2-amino-3-mercaptopropanamido)phenyl)propanoic acid (molecular mass=283.10 Da) at amino acid position 7, is expected to be 102.03 Da. As shown in Table 3 below, the observed average masses of both the WT and UAA proteins are in close agreement with their calculated masses. N-terminal methionine cleavage products of full-length mutant Z domain protein and wt Z-domain protein, and their acetylation products were also observed (see Table 3). The presence of the UAA at amino acid position 7 is posited to impair cleavage of the first methionine, which is mostly “off” in the WT protein and mostly “on” in the mutant variant comprising 2-amino-3-(4-(2-amino-3-mercaptopropanamido)phenyl)propanoic acid. Taken together, SDS PAGE and ESI-MA results indicate that 2-amino-3-(4-(2-amino-3-mercaptopropanamido)phenyl)propanoic acid is incorporated into Z domain with high efficiency and fidelity.

TABLE 3

| | N- | wt Z-domain | | UAA Z-domain | |
|---|---|---|---|---|---|
| First | terminus | Expected | Observed | Expected | Observed |
| Methionine | Acetylated | Mass | Mass | Mass | Mass |
| on | yes | 7971.18 | — | 8073.21 | 8074 |
| on | no | 7928.18 | — | 8030.21 | 8031 |
| | | | | | (major |
| | | | | | peak) |
| off | yes | 7839.99 | 7840 | 7942.02 | — |
| off | no | 7796.99 | 7797 | 7899.02 | 7900 |
| | | | (major | | |
| | | | peak) | | |

### CONJUGATING A PROTEIN COMPRISING 2-AMINO-3-(4-(2-AMINO-3-MERCAPTOPROPANAMIDO)PHENYL)PROPANOIC ACID TO A FLUORESCENT DYE THIOESTER VIA NATIVE CHEMICAL LIGATION

The fluorescent dye fluorescein-MES-thioester (molecular weight=501.03 Da) was conjugated via native chemical ligation (NCL) to purified Z-domain protein comprising the unnatural amino acid (UAA) 2-amino-3-(4-(2-amino-3-mercaptopropanamido)phenyl)propanoic acid at amino acid position 7. In an NCL reaction, the aminothiol moiety that reacts with a thioester moiety to yield a peptide bond at the reaction site is typically provided by a polypeptide comprising an N-terminal cysteine residue. As such, the modifications that can be made to a polypeptide via NCL are limited to the polypeptide's N-terminus. Incorporation of the unnatural amino acid 2-amino-3-(4-(2-amino-3-mercaptopropanamido)phenyl)propanoic acid into a polypeptide permits modifications made via NCL reactions to be made at any amino acid position in a polypeptide.

The purified Z-domain comprising 2-amino-3-(4-(2-amino-3-mercaptopropanamido)phenyl)propanoic acid at amino acid position 7 (UAA Z-domain) was prepared as described above and was resuspended in a buffer comprising 20 mM Na-phosphate, 100 mM NaCl, 1 mM DTT. The pH of this buffer was adjusted to 7.4 at room temperature. The resuspended Z-domain protein was dialyzed at 4° C. (Slidealyzer, Pierce, 0.1-0.5 ml capacity, 3500 MWCO) against 4×1 liter of a buffer comprising 20 mM Na-phosphate, 100 mM NaCl, pH=7.4. The pH of this buffer was adjusted at room temperature.

The fluorescein-MES-thioester (FIG. 5) was prepared from isomer-pure carboxyfluorescein (obtained from EMD biochemicals) and MES-sodium salt. The final product was a MES (methane-ethane-sulfonic acid) thioester, which was purified via HPLC and characterized via LCMS.

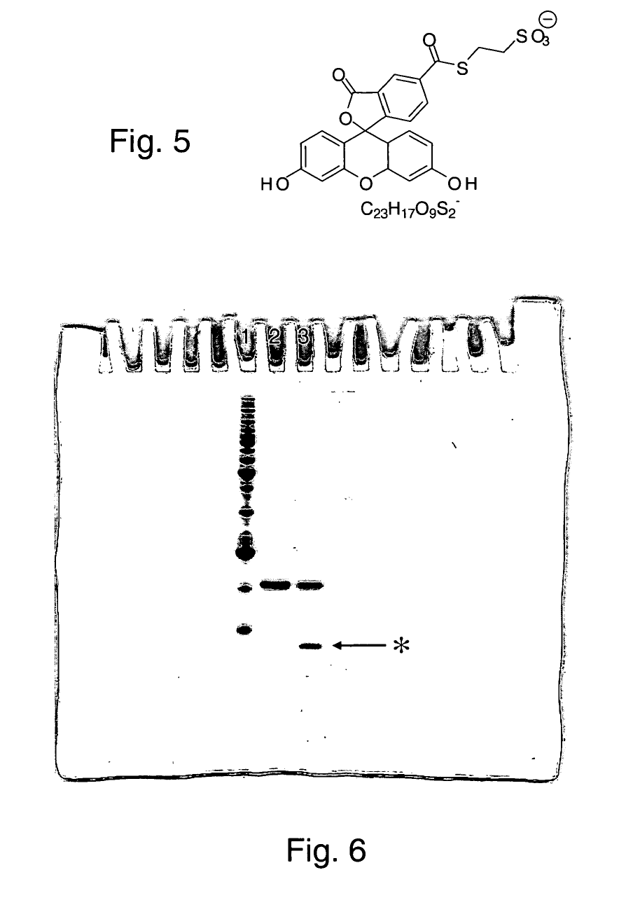

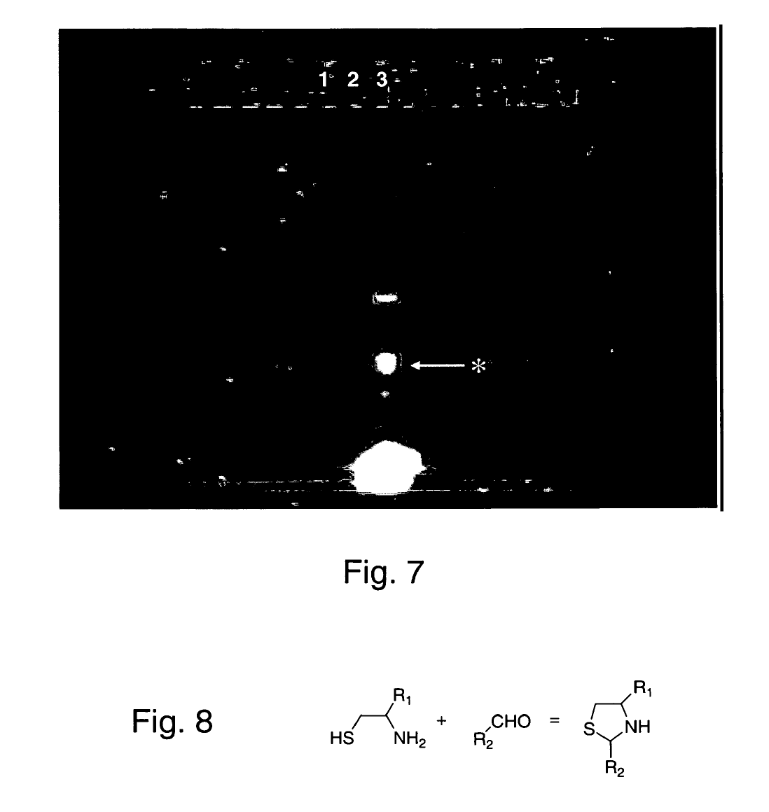

A two-fold molar excess of fluorescein-MES-thioester (in water) was added to the dialyzed UAA Z-domain. The ligation mixture was shaken for 16 hours at 4° C. The crude NCL reaction mixture was run on an SDS gel in non-reducing buffer (MES buffer, available from Invitrogen) and visualized with SimplyBlue™ SafeStain (a Coomassie-like staining method available from Invitrogen), as shown in FIG. 6. Molecular weight markers (Benchmark™, available from Invitrogen) were run in lane 1, unreacted UAA Z-domain protein was run in lane 2, and a sample of the ligation mixture was run in lane 3. The higher molecular weight species in lanes 2 and 3 are UAA-Z domain dimers. The gel in FIG. 6 was also visualized under UV light (FIG. 7). The band in FIG. 7, lane 3 (indicated by *) that corresponds to monomeric UAA-comprising Z domain protein in FIG. 6, lane 3 (also indicated by *) fluoresced under UV light. This result demonstrates that the fluorescent dye fluorescein-MES-thioester was conjugated via NCL to purified Z-domain protein comprising the unnatural amino acid (UAA) 2-amino-3-(4-(2-amino-3-mercaptopropanamido)phenyl)propanoic acid at amino acid position 7.

### SITE SPECIFIC PEGYLATION OF Z-DOMAIN PROTEIN COMPRISING A 2-AMINO-3-(4-(2-AMINO-3-MERCAPTOPROPANAMIDO)PHENYL)PROPANOIC ACID RESIDUE AT AMINO ACID POSITION 7

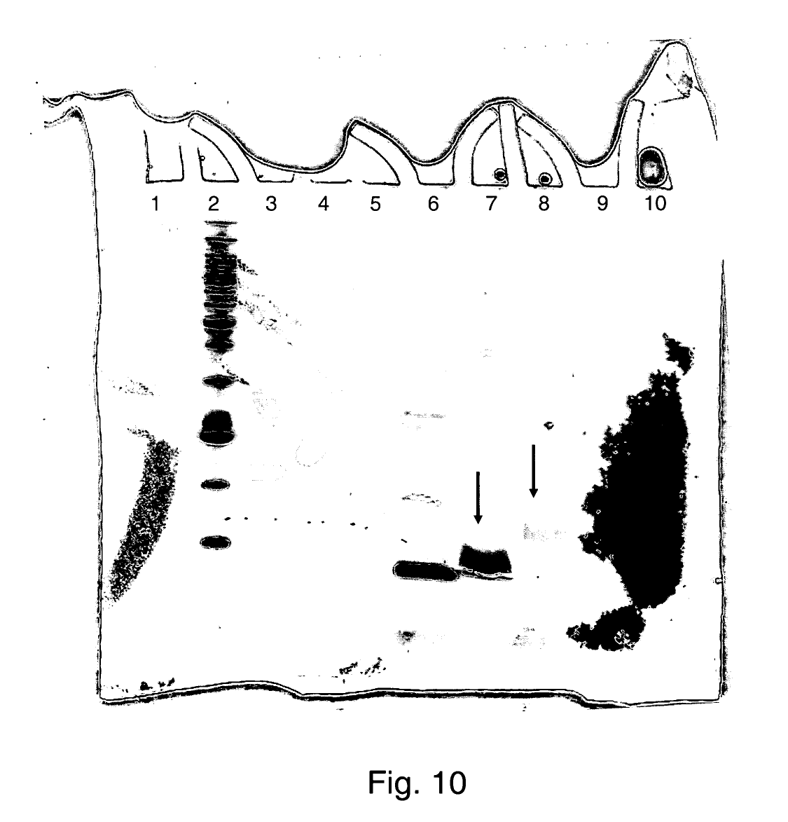

Reaction of an aldehyde with the unnatural amino acid 2-amino-3-(4-(2-amino-3-mercaptopropanamido)phenyl)propanoic acid should produce a stable adduct according to the native chemical ligation (NCL) reaction scheme shown in FIG. 8. Polyethylene glycol (PEG) aldehyde derivatives of two different molecular weights (PEG-2000 and PEG-5000, both purchased from Fluka) were obtained and used in NCL reactions with Z-domain protein comprising a 2-amino-3-(4-(2-amino-3-mercaptopropanamido)phenyl)propanoic acid residue at amino acid position 7 (UAA Z-domain), as described below. Reactions were set up in ratios of 1:1, 1:3, 1:66, or 1:1320 UAA Z-domain: PEG-aldehyde 2000 or in ratios of 1:1, 1:3, 1:26.4 or 1:528 UAA Z-domain: PEG-aldehyde 5000 in aqueous solutions comprising 25 mM HEPES buffer pH=7.4. Each reaction was set up in duplicate. One set of reactions was performed in the presence of 12 mM DTT, and the other was performed in the absence of DTT. All reaction mixtures were shaken overnight (approximately 12-15 hours) at room temperature and run on an SDS gel shown in FIG. 10.

As shown in FIG. 10, molecular weight markers (Benchmark, available from Invitrogen) were run in lane 2, UAA Z-domain was run in lane 6, UAA Z-domain that was reacted with PEG-aldehyde 2000 at a ratio of 1:1320 in the presence of 12 mM DTT was run in lane 7, UAA Z-domain that was reacted with PEG-aldehyde 5000 at a ratio of 1:528 in the presence of 12 mM DTT PEG aldehyde-5000 was run in lane 8. These results indicate that Z-domain comprising 2-amino-3-(4-(2-amino-3-mercaptopropanamido)phenyl)propanoic acid can be PEGylated at the site of the UAA with PEG aldehyde derivatives.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A translation system, comprising:
(a) an unnatural amino acid that comprises a 1,2 aminothiol group;
(b) a first orthogonal aminoacyl-tRNA synthetase (O-RS); and,
(c) a first orthogonal tRNA (O-tRNA);
wherein the first O-RS preferentially aminoacylates the first O-tRNA with the unnatural amino acid.

2. The translation system of claim 1, wherein the unnatural amino acid is 2-amino-3-(4-(2-amino-3-mercaptopropanamido)phenyl)propanoic acid.

3. The translation system of claim 1, wherein the unnatural amino acid that comprises a 1,2 aminothiol group is 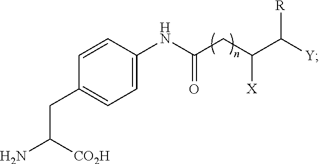
wherein X is NH₂ or SH;
wherein Y is SH when X is NH₂, or NH₂ when X is SH;
wherein R is an H, CH₃, or CH₂CH₃; and,
wherein N is an integer from 0 to 5.

4. The translation system of claim 1, wherein the unnatural amino acid that comprises a 1,2 aminothiol group is 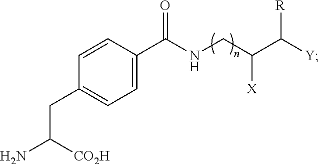
wherein X is NH₂ or SH;
wherein Y is SH when X is NH₂, or NH₂ when X is SH;
wherein R is an H, CH₃, or CH₂CH₃; and,
wherein N is an integer from 1 to 4.

5. The translation system of claim 1, wherein the unnatural amino acid that comprises a 1,2 aminothiol group is 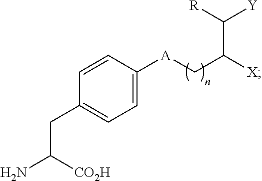
wherein A is O, NH, NCH₃, or S;
wherein X is NH₂ or SH;
wherein Y is SH when X is NH₂, or NH₂ when X is SH;
wherein R is an H, CH₃, or CH₂CH₃; and,
wherein N is an integer from 0 to 6.

6. The translation system of claim 1, wherein the unnatural amino acid that comprises a 1,2 aminothiol group is 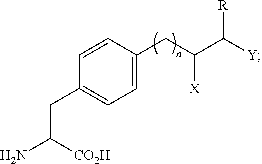
wherein X is NH₂ or SH;
wherein Y is SH when X is NH₂, or NH₂ when X is SH;
wherein R is an H, CH₃, or CH₂CH₃; and,
wherein N is an integer from 1 to 6.

7. The translation system of claim 1, wherein the unnatural amino acid that comprises a 1,2 aminothiol group is 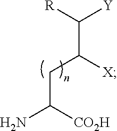
wherein X is NH₂ or SH;
wherein Y is SH when X is NH₂, or NH₂ when X is SH;
wherein R is an H, CH₃, or CH₂CH₃; and,
wherein N is an integer from 1 to 8.

8. The translation system of claim 1, wherein the unnatural amino acid that comprises a 1,2 aminothiol group is 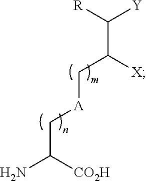
wherein X is NH₂ or SH;
wherein Y is SH when X is NH₂, or NH₂ when X is SH;
wherein R is an H, CH₃, or CH₂CH₃;
wherein N is an integer from 1 to 6;
wherein M is an integer from 1 to 6; and,
wherein A is an O, NH, NCH₃, S,

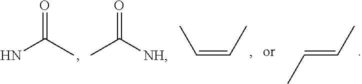

9. The translation system of claim 1, wherein the first O-RS preferentially aminoacylates the first O-tRNA with the unnatural amino acid with an efficiency that is at least 50% of the efficiency observed for a translation system comprising the first O-tRNA, the unnatural amino acid, and an aminoacyl-tRNA synthetase comprising the amino acid sequence of SEQ ID NO: 1.

10. The translation system of claim 1, wherein the first O-RS comprises an amino acid sequence, selected from the group consisting of: an amino acid sequence set forth in SEQ ID NO: 1, and a conservative variant thereof, wherein the conservative variant comprises: an amino acid selected from the group consisting of:
a) glycine at an amino acid position corresponding to amino acid 32 of SEQ ID NO: 1;
b) aspartic acid at an amino acid position corresponding to amino acid 65 of SEQ ID NO: 1;
c) isoleucine at an amino acid position corresponding to amino acid 70 of SEQ ID NO: 1;
d) glutamic acid at an amino acid position corresponding to amino acid 84 of SEQ ID NO: 1;
e) threonine at an amino acid position corresponding to amino acid 108 of SEQ ID NO: 1;
f) tyrosine at an amino acid position corresponding to amino acid 109 of SEQ ID NO: 1;
g) arginine at an amino acid position corresponding to amino acid 114 of SEQ ID NO: 1;
h) glycine at amino acid position corresponding to amino acid 158 of SEQ ID NO: 1;
i) glutamic acid at an amino acid position corresponding to amino acid 162 of SEQ ID NO: 1; and,
j) glycine at an amino acid position corresponding to amino acid 250 of SEQ ID NO: 1.

11. The translation system of claim 1, wherein the first O-tRNA is an amber suppressor tRNA, an ochre suppressor tRNA, an opal suppressor tRNA, or a tRNA that recognizes a four base codon, a rare codon, or a non-coding codon.

12. The translation system of claim 1, wherein the first O-tRNA comprises or is encoded by a polynucleotide sequence set forth in SEQ ID NO: 3.

13. The translation system of claim 1, comprising a nucleic acid encoding a polypeptide of interest, the nucleic acid comprising at least one selector codon, wherein the selector codon is recognized by the first O-tRNA.

14. The translation system of claim 13, wherein the nucleic acid encoding a polypeptide of interest encodes a polypeptide comprising a Z domain, a polypeptide comprising an SH3 domain, or a polypeptide homologous to c-Crk.

15. The translation system of claim 13, further comprising a second O-RS and a second O-tRNA, wherein the second O-RS preferentially aminoacylates the second O-tRNA with a second unnatural amino acid that is different from the unnatural amino acid that comprises the 1,2 aminothiol group, and wherein the second O-tRNA recognizes a selector codon that is different from the selector codon recognized by the first O-tRNA.

16. The translation system of claim 1, wherein the translation system comprises a cell.

17. The translation system of claim 16, wherein the cell is a mammalian cell, an insect cell, a bacterial cell, or an E. coli cell.

18. A method for producing a polypeptide comprising at least one unnatural amino acid that comprises a 1,2 aminothiol group at a selected position, the method comprising:
(a) providing a translation system comprising:
(i) an unnatural amino acid that comprises a 1,2-aminothiol group;
(ii) a first orthogonal aminoacyl-tRNA synthetase (O-RS);
(iii) a first orthogonal tRNA (O-tRNA), wherein the first O-RS preferentially aminoacylates the first O-tRNA with the unnatural amino acid that comprises a 1,2-aminothiol group; and,
(iv) a nucleic acid of interest encoding the polypeptide of interest, wherein the nucleic acid comprises at least one selector codon that is recognized by the first O-tRNA; and,
(b) incorporating the unnatural amino acid that comprises a 1,2 aminothiol group at the selected position in the polypeptide during translation of the polypeptide in response to the selector codon, thereby producing the polypeptide comprising the unnatural amino acid that comprises a 1,2 aminothiol group at the selected position.

19-24. (canceled)

25. A composition comprising a polynucleotide encoding an orthogonal aminoacyl tRNA synthetase (O-RS) that preferentially aminoacylates a cognate orthogonal tRNA (O-tRNA) with an unnatural amino acid that comprises a 1,2 aminothiol group.

26-30. (canceled)

31. A method of producing an orthogonal aminoacyl-tRNA synthetase that preferentially aminoacylates an O-tRNA with a 2-amino-3-(4-(2-amino-3-mercaptopropanamido)phenyl)propanoic acid, the method comprising:
a) mutating a wild-type aminoacyl-tRNA synthetase; and,
b) selecting a resulting O-RS that preferentially aminoacylates the first O-tRNA with the 2-amino-3-(4-(2-amino-3-mercaptopropanamido)phenyl)propanoic acid, thereby providing the first O-RS.

32. A method of synthesizing 2-amino-3-(4-(2-amino-3-mercaptopropanamido)phenyl)-propanoic acid, the method comprising:
a) dissolving N-(tert-butoxycarbonyl)-S-(triphenylmethyl)cysteine, 1-(3-dimethylaminopropyl)-3-ethylcarbodiimide hydrochloride, and 1-hydroxybenzotriazole hydrate in anhydrous dimethylformamide to produce solution 1,
b) adding N,N-diisopropylethylamine to solution 1 to produce solution 2,
c) adding N-(tert-butoxycarbonyl)-4-aminophenylalanine to solution 2 to produce solution 3,
d) drying solution 3 to produce residue 1,
e) purifying residue 1 to produce solid 1,
f) dissolving solid 1 in a mixture comprising trifluoroacetic acid, triisopropylsilane, thioanisole and water to produce solution 4,
g) drying solution 4 to produce residue 2, and;
h) purifying residue 2, which comprises the 2-amino-3-(4-(2-amino-3-mercaptopropan-amido)phenyl)-propanoic acid.

33-37. (canceled)
